(19) United States
(12) Patent Application Publication
(10) Pub. No.: US 2022/0192989 A1
(43) Pub. Date: Jun. 23, 2022

(54) METHODS AND COMPOSITIONS FOR TREATING RESPIRATORY ARRHYTHMIAS

(71) Applicant: Meter Health, Inc., Manchester, CT (US)

(72) Inventors: Adam Kievman, Manchester, CT (US); Mallory Kievman, Manchester, CT (US); Abraham Friedman, West Hartford, CT (US); Bartholomew J. Bacak, Rochester, NY (US)

(21) Appl. No.: 17/604,691

(22) PCT Filed: Apr. 17, 2020
(86) PCT No.: PCT/US2020/028708

§ 371 Date: Oct. 18, 2021

Related U.S. Application Data
(60) Provisional application No. 62/835,691, filed on Apr. 18, 2019.

(51) Int. Cl. A61K 9/20; A61K 9/00; A61K 31/047; A61K 31/194

(52) U.S. Cl. CPC A61K 9/2018; A61K 9/0056; A61K 9/2095; A61K 31/194; A61K 31/047

(57) ABSTRACT

The present disclosure relates to compositions to perturb neural circuits and/or the brainstem respiratory network and methods of use thereof.

### CROSS REFERENCE TO RELATED APPLICATIONS

This application claims the benefit of U.S. Provisional Patent Application No. 62/835,691 filed Apr. 18, 2019 entitled “Methods and Compositions for Treating Respiratory Arrhythmias”, the contents of which are herein incorporated by reference in its entirety.

### FIELD

The present disclosure relates to compositions and methods to perturb neural circuits and/or the brainstem respiratory network. These compositions and methods may be used to treat and/or prevent respiratory arrythmias (e.g., chronic hiccups, or sporadic hiccups), nausea, vomiting, salivary gland disorders, and/or xerostomia.

### BACKGROUND

Respiratory disorders such as respiratory arrhythmias have been studied and evaluated for well over a century. Respiratory arrhythmias are irregular breathing patterns which may be caused by numerous disease, disorders and/or conditions or they may be a side-effect from different medications. Treatment of respiratory arrhythmias has included drugs (e.g., Thorazine) as well as folk remedies, off-label drugs, massage, and increasing the amount of carbon dioxide inhaled. But none of these methods have been able to systematically and sufficiently treat respiratory arrhythmias. Therefore, the present disclosure has provided composition and methods to treat respiratory arrhythmias by perturbing the signals to the neural circuits in the brain.

### SUMMARY

The present disclosure provides pharmaceutical compositions which may include one or more perturbation stimuli such as, but not limited to, chemical stimuli, external electrical stimuli, and external mechanical stimuli. In some aspects, the perturbation stimulation is a chemical stimuli such as, but not limited to, Acetic acid, Ascorbic acid, Citric acid, D-Malic acid, L-Malic acid, D/L-Malic acid, Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. In one aspect, the chemical stimuli is D-Malic acid and may optionally include Acetic acid a chemical stimuli. In another aspect, the chemical stimuli is L-Malic acid and may optionally include Acetic acid a chemical stimuli. In yet another aspect, the chemical stimuli is D/L-Malic acid and may optionally include Acetic acid a chemical stimuli.

The pharmaceutical composition may also include one or more excipients. Non-limiting examples of excipients include solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. In one aspect, the excipient is a sugar or sugar alcohol such as, but not limited to, dextrose, dextrin, glucose, isomaltitol, lactitol, lactose, mannitol, sorbitol, sucrose, and xylitol. In one aspect, the sugar alcohol is mannitol.

The present disclosure provides an orally disintegrating tablet (ODT) which may include one or more perturbation stimuli such as, but not limited to, chemical stimuli, external electrical stimuli, and external mechanical stimuli. In some aspects, the perturbation stimulation is a chemical stimuli such as, but not limited to, Acetic acid, Ascorbic acid, Citric acid, D-Malic acid, L-Malic acid, D/L-Malic acid, Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. In one aspect, the chemical stimuli is D-Malic acid and may optionally include Acetic acid a chemical stimuli. In another aspect, the chemical stimuli is L-Malic acid and may optionally include Acetic acid a chemical stimuli. In yet another aspect, the chemical stimuli is D/L-Malic acid and may optionally include Acetic acid a chemical stimuli.

The ODT may also include one or more excipients. Non-limiting examples of excipients include solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. In one aspect, the excipient is a sugar or sugar alcohol such as, but not limited to, dextrose, dextrin, glucose, isomaltitol, lactitol, lactose, mannitol, sorbitol, sucrose, and xylitol. In one aspect, the sugar alcohol is mannitol.

Provided herein are methods of treating a disease, disorder or condition related to respiratory arrythmia by administering a therapeutically effective amount of the pharmaceutical composition or ODT to a subject in need thereof. The respiratory arrythmia may be caused by a disease, disorder or condition such as, but not limited to, cancer, advanced cancer, apnea, orthopnea, dyspnea, hyperpnea, hyperventilation, hypoventilation, tachypnea, Kussmaul respiration, Cheyne-Stokes respiration, sighing respiration, Biot respiration, apneustic breathing, central neurogenic hyperventilation, central neurogenic hypoventilation, agonal breathing, allergies, multiple sclerosis, Parkinson disease, Trauma, Postpolio syndrome, Amyotrophic Lateral Sclerosis, Guillain-Barré syndrome, Charcot-Marie-Tooth disease, Myasthenia gravis, Botulism, Duchenne muscular dystrophy, Polymyositis/dermatomyositis, Postparalysis myopathy, asthma, chronic obstructive pulmonary disease (COPD) and interstitial lung disease, anxiety related disorders, gastroesophageal reflux disease (GERD), panic disorder, Rett syndrome, effects of post-surgery recovery, weight loss, and insomnia. In one aspect, the disease, disorder or condition related to respiratory arrythmia is chronic hiccups, sporadic hiccups. The effect of chronic hiccups, sporadic hiccups may be reduced so a subject does not hiccup for a time period such as, but not limited to, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5days) and more than 120 hours ( 5 days).

### DETAILED DESCRIPTION

I. Respiration and Respiratory Arrythmia

In general, respiration is controlled by neural circuits which are spatially organized in the brainstem respiratory network (BRN) (Molkov et al. J Neurophysiol. 2010 Nov. 104( 5): 2713- 2729; Molkov et al. Frontiers in Neural Circuits. 2013 Feb. 7( 16). DOI: 10.3389/fncir. 2013.00016; Molkov et al. PLOS ONE 2014. 9( 10), e 109894). These neural circuits are “multi-stable” meaning they able to exist in any one of multiple, qualitatively different, states dependent on input signals sent to the BRN. These signals may include differences in membrane potential (voltage, V) from the mechanical feedback provided by pulmonary mechanoreceptors on lung volume and chemical feedback from peripheral and central chemoreceptors on the levels of carbon dioxide and oxygen in the blood and brain tissue.

Changes (e.g., perturbations) in the signals provided to the BRN can cause the neural circuits to switch between states. For example, in one state of the neural circuit, respiration has a normal respiratory rhythm. “Normal respiratory rhythm” or “eupnea” is defined as a period of inspiration (e.g., inhalation) followed by a period of expiration (e.g., exhalation) where the period of expiration is longer than the period of inspiration. Inspiratory neurons are the neurons which are active during inspiration and expiratory neurons are the neurons active during expiration.

A perturbation to the neural circuit and/or the BRN may switch the state of the neural circuit to a state where respiration includes periods of normal respiratory rhythm separated by at least one period of rapid and high amplitude inspirations (e.g., hiccups). When the neural circuit is in a state that causes respiration which is different than the normal respiratory rhythm, this form of respiration is referred to as “respiratory arrythmia.” The BRN therefore has the capacity to exhibit both a normal respiratory rhythm and a respiratory arrythmia for a given set of signals. The neural circuits producing respiration in a normal respiratory rhythm may be perturbed so that the neural circuits produce respiratory arrythmias. Additionally, neural circuits producing respiratory arrythmias may also be perturbed in order to revert respiration back to the normal respiratory rhythm.

Described herein are compositions and methods to perturb neural circuits and/or the BRN in order to treat and/or prevent respiratory arrythmia.

Respiratory arrythmia may be caused a wide-array of diseases, disorders and/or conditions related to respiratory irregularities. Non-limiting examples of diseases, disorders and/or conditions associated with respiratory irregularities include cancer, advanced cancer, apnea, orthopnea, dyspnea, hyperpnea, hyperventilation, hypoventilation, tachypnea, Kussmaul respiration, Cheyne-Stokes respiration, sighing respiration, Biot respiration, apneustic breathing, central neurogenic hyperventilation, central neurogenic hypoventilation, agonal breathing, allergies, multiple sclerosis, Parkinson disease, Trauma, Postpolio syndrome, Amyotrophic Lateral Sclerosis, Guillain-Barré syndrome, Charcot-Marie-Tooth disease, Myasthenia gravis, Botulism, Duchenne muscular dystrophy, Polymyositis/dermatomyositis, Postparalysis myopathy, asthma, chronic obstructive pulmonary disease (COPD) and interstitial lung disease, anxiety related disorders, gastroesophageal reflux disease (GERD), panic disorder, Rett syndrome, effects of post-surgery recovery, weight loss, and insomnia.

Additional respiratory arrythmia may be caused as a side effect of medications (e.g., dose of medication) or treatment procedures for a disease, disorder and/or condition.

Besides a change in the normal respiratory rhythm, signs and symptoms of respiratory arrythmia included, but are not limited to, headache, air hunger, sighing/yawning, tight chest, asthma, panic attacks, excess wind, cramps/tremors, pins and needles in fingers and toes, dizziness/fainting, cough, dry throat, palpitations (noticeable heartbeats), chest pain, anxiety, weakness, and unreal feelings.

Computer Modeling of Rhythmic Behaviors such as Respiration

In order to better to understand the rhythmic behaviors of the BRN and other networks in the human body, various models have been developed to study the signals which are fed into central pattern generators (CPGs) which produce motor outputs and the signals that are fed back into the CPG to regulate the motor output. One example of a CPG is the respiratory CPG in the BRN which uses information from the neural circuits to regulate respiration.

One model was developed to explore the gait in limbed animals and the pattern generation circuits which control the gait (Molkov et al. ( 2015) PLoS Comput Biol 11 ( 5): e 1004270.doi: 10.1371/journal.pcbi. 1004270). Bacak et al. developed a model to explore mixed-mode oscillations (MMOs) where there are alternations between large amplitude (LA) and small amplitude (SA) oscillations (Bacak et al. eLife 2016;5:e 1343. DOI: 10.7554/eLife. 13403). Additionally, modeling and qualitative analysis was described by Rubin et al. to explore the interactions between two neural oscillators which are known to be involved in the control of breathing in mammals (Rubin et al. J Comput Neurosci. 2011 June; 30( 3): 607- 632. DOI: 10.1007/s 10827- 010- 0281- 0).

### II. Perturbations

The signals to the BRN and/or neural circuits may be altered by perturbed by a stimulus in order to have the neural circuits be in a state to produce a normal respiratory rhythm or to the neural circuits from a state of respiratory arrythmia back to a state of the normal respiratory rhythm. The perturbing stimulus may be direct or indirect to the BRN or the neural circuits. Perturbation stimuli may have effects on the brain steam, hippocampus, airways, lungs, joint proprioceptors and/or peripheral chemoreceptors which can change the signal to the BRN and neural circuits which can alter the respiratory rhythm. Examples of perturbation stimuli categories include, but are not limited to, chemical stimuli (e.g., small molecules, lipids, fats, nucleic acids, peptides, proteins), mechanical stimuli (e.g. applied force or pressure on deformation sensing receptors), electrical stimuli, or other stimuli.

### Chemical Stimuli

In some embodiments, respiratory arrythmia may be treated using chemical stimuli alone or in combination with the compositions described herein. The chemical stimuli may come from within the body (which is referred to as “internal chemical stimuli”) or the chemical stimuli may come from a source outside of the body (which is referred to as “external chemical stimuli”).

### Internal Chemical Stimuli

In some embodiments, respiratory arrythmia may be treated using internal chemical stimuli alone or in combination with the compositions described herein. As a non-limiting example, the respiratory arrythmia is chronic hiccups. As a non-limiting example, the respiratory arrythmia is sporadic hiccups.

An example of internal chemical stimuli is elevation of carbon dioxide (CO₂) levels in the body as CO₂ is a potent activator of inspiratory drive in mammals. An increase the level of CO₂ in mammals can be caused by volitional methods (e.g., breath holding), or nonvolitional methods (e.g., renal or respiratory disease).

### External Chemical Stimuli

In some embodiments, respiratory arrythmia may be treated using external chemical stimuli alone or in combination with the composition described herein. As a non-limiting example, the respiratory arrythmia is chronic hiccups. As a non-limiting example, the respiratory arrythmia is sporadic hiccups.

In some embodiments, an external chemical stimuli may be used alone or in combination with the compositions described herein to perturb the signals in the BRN or neural circuits.

In some embodiments, the external chemical stimuli is increasing the concentration of CO₂ inhaled on inspiration.

In some embodiments, the external chemical stimuli is acidic agents. The acidic agents may be organic or inorganic. Non-limiting examples of acidic agents include Acetic acid, Aconitic Acid, Adipic acid, Alginic acid, L-Ascorbic acid, Amino acids (all), Benzoic Acid, Caprylic Acid, Cholic acid, Citric acid, Dehydroacetic acid, Desoxycholic acid, Erythorbic acid (D-isoascorbic acid), Folic acid, Formic acid, Fumaric acid, L-Glutamic acid, L-Glutamic acid hydrochloride, Glycocholic acid, Hydrochloric acid, D(-)-Lactic acid, Lactic acid, L(+)-lactic acid, Linoleic acid, Malic acid, L-Malic acid, a mixture of D- and L-Malic acid, Maleic acid, Niacin (nicotinic acid), Oleic acid, Pectinic acid, Phosphoric acid, L(+)-potassium acid tartrate, Propionic acid, Sodium acid pyrophosphate, Sorbic acid, Stearic acid, Succinic acid, Sulfamic acid, Sulfuric Acid, Tannic acid, L(+)-tartaric acid, Taurocholic acid, Thiodipropionic acid, and their calcium, ferrous, magnesium, potassium, and sodium salts.

In some embodiments, the external chemical stimuli is a food product. Non-limiting examples of food products include citrus fruits (e.g., lemon and limes), coffee, sour milk products (e.g., yogurt, cheese, and sour cream), and fermented products (e.g., wine, vinegar, pickles, sauerkraut, and soy sauce).

In some embodiments, the external chemical stimuli is a chemical agent. Non-limiting examples of chemical agents include GABA receptor agonist (e.g., baclofen), vanilloid receptor subtype 1 (VR1) antagonists, Alpha2delta (α2δ) ligands, NK 1 receptor antagonist, NK 3 receptor antagonist, TTX-sensitive sodium channels (riluzole), opioids, dopamine antagonists and anti-psychotic medications.

In some embodiments, the external chemical stimuli is an agent which is converted to an acid after contact with water. Non-limiting examples include a salt form of an acid. Another non-limiting example is sodium acetate which, when placed in water, will have at least a portion become acetic acid.

### External Electrical Stimuli

In some embodiments, respiratory arrythmia may be treated using external electrical stimuli alone or in combination with the composition described herein. As a non-limiting example, the respiratory arrythmia is chronic hiccups. As a non-limiting example, the respiratory arrythmia is sporadic hiccups.

In some embodiments, the external electrical stimulation is to the medullary reticular formation.

In some embodiments, the external electrical stimulation is direct simulation to the muscles and/or nerves of the diaphragm. As a non-limiting example, the external electrical simulation is NeuRx® diaphragm pacing system (DPS) from Synapse Biomedical Inc. which includes (a) four PermaLoc® electrodes are implanted in the diaphragm to provide direct muscle stimulation, (b) a fifth electrode is implanted under the skin to ground the system and complete the circuit, (c) an electrode connector to group the electrodes into a socket, (d) an external pulse generator (EPG) and a removable cable to connect the electrode socket to the EPG.

In some embodiments, the external electrical stimulation is by phrenic nerve stimulation via electrodes in the chest and/or neck. As a non-limiting example, the phrenic nerve stimulation device is a phrenic nerve stimulator from Avery Biomedical Devices.

In some embodiments, the external electrical stimulation is to the forelimb flexor muscles (Potts et al. Respiratory rhythm entrainment by somatic afferent stimulation. J Neuroscience 2005).

In some embodiments, the external electrical stimulation is to the glottis. External Mechanical stimuli

In some embodiments, respiratory arrythmia may be treated using external mechanical stimuli alone or in combination with the composition described herein. As a non-limiting example, the respiratory arrythmia is chronic hiccups. As a non-limiting example, the respiratory arrythmia is sporadic hiccups.

In some embodiments, the external mechanical stimulation is stimulation to the pharynx.

In some embodiments, the external mechanical stimulation is a digital rectal massage.

In some embodiments, the external mechanical stimulation is to the glottis.

### III. Formulations

### Active Ingredients

In some embodiments, the active ingredient is a chemical stimuli. The chemical stimuli may be selected from, but is not limited to Acetic acid, Ascorbic acid, Citric acid, Malic acid (e.g., D-Malic acid, L-Malic acid, D/L-Malic acid), Maleic acid, Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, at least one of the chemical stimuli is an acid such as Citric acid, Malic acid, Maleic acid, Fumaric acid, and Ascorbic acid. As a non-limiting example, at least one of the chemical stimuli is a food acid such as vinegar, citric acid, tartaric acid, malic acid, folic acid, fumaric acid, and lactic acid. As a non-limiting example, at least one of the chemical stimuli is D-Malic acid. As another non-limiting example, at least one of the chemical stimuli is L-Malic acid. As another non-limiting example, at least one of the chemical stimuli is a combination of D-Malic acid and L-Malic acid (which may be referred to as “D/L-Malic acid”).

In some embodiments, the active ingredient is a chemical stimuli and at least one of the chemical stimuli is D-Malic acid.

In some embodiments, the active ingredient is a chemical stimuli and at least one of the chemical stimuli is L-Malic acid.

In some embodiments, the active ingredient is a chemical stimuli and at least one of the chemical stimuli is a combination of D-Malic acid and L-Malic acid (which may be referred to as “D/L-Malic acid”). The D/L-Malic acid may include between 1% to 99% of D- or L-Malic acid. The D/L-Malic acid may include D-Malic acid at a percentage of 1% and include L-Malic acid at a percentage of 99%. The D/L-Malic acid may include D-Malic acid at a percentage of 2% and include L-Malic acid at a percentage of 98%. The D/L-Malic acid may include D-Malic acid at a percentage of 3% and include L-Malic acid at a percentage of 97%. The D/L-Malic acid may include D-Malic acid at a percentage of 4% and include L-Malic acid at a percentage of 96%. The D/L-Malic acid may include D-Malic acid at a percentage of 5% and include L-Malic acid at a percentage of 95%. The D/L-Malic acid may include D-Malic acid at a percentage of 6% and include L-Malic acid at a percentage of 94%. The D/L-Malic acid may include D-Malic acid at a percentage of 7% and include L-Malic acid at a percentage of 93%. The D/L-Malic acid may include D-Malic acid at a percentage of 8% and include L-Malic acid at a percentage of 92%. The D/L-Malic acid may include D-Malic acid at a percentage of 9% and include L-Malic acid at a percentage of 91%. The D/L-Malic acid may include D-Malic acid at a percentage of 10% and include L-Malic acid at a percentage of 90%. The D/L-Malic acid may include D-Malic acid at a percentage of 11% and include L-Malic acid at a percentage of 89%. The D/L-Malic acid may include D-Malic acid at a percentage of 12% and include L-Malic acid at a percentage of 88%. The D/L-Malic acid may include D-Malic acid at a percentage of 13% and include L-Malic acid at a percentage of 87%. The D/L-Malic acid may include D-Malic acid at a percentage of 14% and include L-Malic acid at a percentage of 86%. The D/L-Malic acid may include D-Malic acid at a percentage of 15% and include L-Malic acid at a percentage of 85%. The D/L-Malic acid may include D-Malic acid at a percentage of 16% and include L-Malic acid at a percentage of 84%. The D/L-Malic acid may include D-Malic acid at a percentage of 17% and include L-Malic acid at a percentage of 83%. The D/L-Malic acid may include D-Malic acid at a percentage of 18% and include L-Malic acid at a percentage of 82%. The D/L-Malic acid may include D-Malic acid at a percentage of 19% and include L-Malic acid at a percentage of 81%. The D/L-Malic acid may include D-Malic acid at a percentage of 20% and include L-Malic acid at a percentage of 80%. The D/L-Malic acid may include D-Malic acid at a percentage of 21% and include L-Malic acid at a percentage of 79%. The D/L-Malic acid may include D-Malic acid at a percentage of 22% and include L-Malic acid at a percentage of 78%. The D/L-Malic acid may include D-Malic acid at a percentage of 23% and include L-Malic acid at a percentage of 77%. The D/L-Malic acid may include D-Malic acid at a percentage of 24% and include L-Malic acid at a percentage of 76%. The D/L-Malic acid may include D-Malic acid at a percentage of 25% and include L-Malic acid at a percentage of 75%. The D/L-Malic acid may include D-Malic acid at a percentage of 26% and include L-Malic acid at a percentage of 74%. The D/L-Malic acid may include D-Malic acid at a percentage of 27% and include L-Malic acid at a percentage of 73%. The D/L-Malic acid may include D-Malic acid at a percentage of 28% and include L-Malic acid at a percentage of 72%. The D/L-Malic acid may include D-Malic acid at a percentage of 29% and include L-Malic acid at a percentage of 71%. The D/L-Malic acid may include D-Malic acid at a percentage of 30% and include L-Malic acid at a percentage of 70%. The D/L-Malic acid may include D-Malic acid at a percentage of 31% and include L-Malic acid at a percentage of 69%. The D/L-Malic acid may include D-Malic acid at a percentage of 32% and include L-Malic acid at a percentage of 68%. The D/L-Malic acid may include D-Malic acid at a percentage of 33% and include L-Malic acid at a percentage of 67%. The D/L-Malic acid may include D-Malic acid at a percentage of 34% and include L-Malic acid at a percentage of 66%. The D/L-Malic acid may include D-Malic acid at a percentage of 35% and include L-Malic acid at a percentage of 65%. The D/L-Malic acid may include D-Malic acid at a percentage of 36% and include L-Malic acid at a percentage of 64%. The D/L-Malic acid may include D-Malic acid at a percentage of 37% and include L-Malic acid at a percentage of 63%. The D/L-Malic acid may include D-Malic acid at a percentage of 38% and include L-Malic acid at a percentage of 62%. The D/L-Malic acid may include D-Malic acid at a percentage of 39% and include L-Malic acid at a percentage of 61%. The D/L-Malic acid may include D-Malic acid at a percentage of 40% and include L-Malic acid at a percentage of 60%. The D/L-Malic acid may include D-Malic acid at a percentage of 41% and include L-Malic acid at a percentage of 59%. The D/L-Malic acid may include D-Malic acid at a percentage of 42% and include L-Malic acid at a percentage of 58%. The D/L-Malic acid may include D-Malic acid at a percentage of 43% and include L-Malic acid at a percentage of 57%. The D/L-Malic acid may include D-Malic acid at a percentage of 44% and include L-Malic acid at a percentage of 56%. The D/L-Malic acid may include D-Malic acid at a percentage of 45% and include L-Malic acid at a percentage of 55%. The D/L-Malic acid may include D-Malic acid at a percentage of 46% and include L-Malic acid at a percentage of 54%. The D/L-Malic acid may include D-Malic acid at a percentage of 47% and include L-Malic acid at a percentage of 53%. The D/L-Malic acid may include D-Malic acid at a percentage of 48% and include L-Malic acid at a percentage of 52%. The D/L-Malic acid may include D-Malic acid at a percentage of 49% and include L-Malic acid at a percentage of 51%. The D/L-Malic acid may include D-Malic acid at a percentage of 50% and include L-Malic acid at a percentage of 50%. The D/L-Malic acid may include D-Malic acid at a percentage of 51% and include L-Malic acid at a percentage of 49%. The D/L-Malic acid may include D-Malic acid at a percentage of 52% and include L-Malic acid at a percentage of 48%. The D/L-Malic acid may include D-Malic acid at a percentage of 53% and include L-Malic acid at a percentage of 47%. The D/L-Malic acid may include D-Malic acid at a percentage of 54% and include L-Malic acid at a percentage of 46%. The D/L-Malic acid may include D-Malic acid at a percentage of 55% and include L-Malic acid at a percentage of 45%. The D/L-Malic acid may include D-Malic acid at a percentage of 56% and include L-Malic acid at a percentage of 44%. The D/L-Malic acid may include D-Malic acid at a percentage of 57% and include L-Malic acid at a percentage of 43%. The D/L-Malic acid may include D-Malic acid at a percentage of 58% and include L-Malic acid at a percentage of 42%. The D/L-Malic acid may include D-Malic acid at a percentage of 59% and include L-Malic acid at a percentage of 41%. The D/L-Malic acid may include D-Malic acid at a percentage of 60% and include L-Malic acid at a percentage of 40%. The D/L-Malic acid may include D-Malic acid at a percentage of 61% and include L-Malic acid at a percentage of 39%. The D/L-Malic acid may include D-Malic acid at a percentage of 62% and include L-Malic acid at a percentage of 38%. The D/L-Malic acid may include D-Malic acid at a percentage of 63% and include L-Malic acid at a percentage of 37%. The D/L-Malic acid may include D-Malic acid at a percentage of 64% and include L-Malic acid at a percentage of 36%. The D/L-Malic acid may include D-Malic acid at a percentage of 65% and include L-Malic acid at a percentage of 35%. The D/L-Malic acid may include D-Malic acid at a percentage of 66% and include L-Malic acid at a percentage of 34%. The D/L-Malic acid may include D-Malic acid at a percentage of 67% and include L-Malic acid at a percentage of 33%. The D/L-Malic acid may include D-Malic acid at a percentage of 68% and include L-Malic acid at a percentage of 32%. The D/L-Malic acid may include D-Malic acid at a percentage of 69% and include L-Malic acid at a percentage of 31%. The D/L-Malic acid may include D-Malic acid at a percentage of 70% and include L-Malic acid at a percentage of 30%. The D/L-Malic acid may include D-Malic acid at a percentage of 71% and include L-Malic acid at a percentage of 29%. The D/L-Malic acid may include D-Malic acid at a percentage of 72% and include L-Malic acid at a percentage of 28%. The D/L-Malic acid may include D-Malic acid at a percentage of 73% and include L-Malic acid at a percentage of 27%. The D/L-Malic acid may include D-Malic acid at a percentage of 74% and include L-Malic acid at a percentage of 26%. The D/L-Malic acid may include D-Malic acid at a percentage of 75% and include L-Malic acid at a percentage of 25%. The D/L-Malic acid may include D-Malic acid at a percentage of 76% and include L-Malic acid at a percentage of 24%. The D/L-Malic acid may include D-Malic acid at a percentage of 77% and include L-Malic acid at a percentage of 23%. The D/L-Malic acid may include D-Malic acid at a percentage of 78% and include L-Malic acid at a percentage of 22%. The D/L-Malic acid may include D-Malic acid at a percentage of 79% and include L-Malic acid at a percentage of 21%. The D/L-Malic acid may include D-Malic acid at a percentage of 80% and include L-Malic acid at a percentage of 20%. The D/L-Malic acid may include D-Malic acid at a percentage of 81% and include L-Malic acid at a percentage of 19%. The D/L-Malic acid may include D-Malic acid at a percentage of 82% and include L-Malic acid at a percentage of 18%. The D/L-Malic acid may include D-Malic acid at a percentage of 83% and include L-Malic acid at a percentage of 17%. The D/L-Malic acid may include D-Malic acid at a percentage of 84% and include L-Malic acid at a percentage of 16%. The D/L-Malic acid may include D-Malic acid at a percentage of 85% and include L-Malic acid at a percentage of 15%. The D/L-Malic acid may include D-Malic acid at a percentage of 86% and include L-Malic acid at a percentage of 14%. The D/L-Malic acid may include D-Malic acid at a percentage of 87% and include L-Malic acid at a percentage of 13%. The D/L-Malic acid may include D-Malic acid at a percentage of 88% and include L-Malic acid at a percentage of 12%. The D/L-Malic acid may include D-Malic acid at a percentage of 89% and include L-Malic acid at a percentage of 11%. The D/L-Malic acid may include D-Malic acid at a percentage of 90% and include L-Malic acid at a percentage of 10%. The D/L-Malic acid may include D-Malic acid at a percentage of 91% and include L-Malic acid at a percentage of 9%. The D/L-Malic acid may include D-Malic acid at a percentage of 92% and include L-Malic acid at a percentage of 8%. The D/L-Malic acid may include D-Malic acid at a percentage of 93% and include L-Malic acid at a percentage of 7%. The D/L-Malic acid may include D-Malic acid at a percentage of 94% and include L-Malic acid at a percentage of 6%. The D/L-Malic acid may include D-Malic acid at a percentage of 95% and include L-Malic acid at a percentage of 5%. The D/L-Malic acid may include D-Malic acid at a percentage of 96% and include L-Malic acid at a percentage of 4%. The D/L-Malic acid may include D-Malic acid at a percentage of 97% and include L-Malic acid at a percentage of 3%. The D/L-Malic acid may include D-Malic acid at a percentage of 98% and include L-Malic acid at a percentage of 2%. The D/L-Malic acid may include D-Malic acid at a percentage of 99% and include L-Malic acid at a percentage of 1%.

In some embodiment, the active ingredient is a combination of more than one chemical stimuli. As a non-limiting example, the active ingredient is Acetic acid and Ascorbic acid, Acetic acid and Malic acid (e.g., D-Malic acid, L-Malic acid, D/L-Malic acid), Acetic acid and Citric acid, Acetic acid and Malic acid (e.g., D-Malic acid, L-Malic acid, D/L-Malic acid) and Citric acid, Acetic acid and Citric acid and Ascorbic acid, Malic acid (e.g., D-Malic acid, L-Malic acid, D/L-Malic acid) and Citric acid, L-Malic acid and Citric acid, Malic acid (e.g., D-Malic acid, L-Malic acid, D/L-Malic acid) and Fumaric acid, Malic acid (e.g., D-Malic acid, L-Malic acid, D/L-Malic acid) and Ascorbic acid, Acetic acid and Citric acid and Ascorbic acid and Niacin, Acetic acid and Phosphoric acid, Malic acid (e.g., D-Malic acid, L-Malic acid, D/L-Malic acid) and Phosphoric acid, Sodium malate and Sodium citrate, Sodium acetate and Sodium citrate, Potassium malate and Sodium ascorbate, Sodium malate and Calcium citrate, Sodium malate and Potassium citrate, Sodium acetate and Potassium citrate, Sodium acetate and Citric acid, Sodium malate and Acetic acid, Sodium malate and Phosphoric acid, Sodium malate and Citric acid, Calcium phosphate and Hydrochloric acid, Potassium malate and Acetic acid. As a non-limiting example, the chemical stimuli is D-Malic acid and Acetic acid. As another non-limiting example, the chemical stimuli is L-Malic acid and Acetic acid. As another non-limiting example, the chemical stimuli is D/L-Malic acid and Acetic acid. As a non-limiting example, the chemical stimuli is D-Malic acid and Mannitol. As another non-limiting example, the chemical stimuli is L-Malic acid and Mannitol. As another non-limiting example, the chemical stimuli is D/L-Malic acid and Mannitol.

In some embodiment, the active ingredient is any chemical stimuli described herein or known in the art alone or in combination with each other or with other agents.

In some embodiments, the percentage range of the active ingredients, or each active ingredient if there is more than one, in the formulation is between 20% and 80%. The percentage range of the active ingredient in the formulation is between 20% and 25%. The percentage range of the active ingredient in the formulation is between 20% and 30%. The percentage range of the active ingredient in the formulation is between 20% and 35%. The percentage range of the active ingredient in the formulation is between 20% and 40%. The percentage range of the active ingredient in the formulation is between 20% and 45%. The percentage range of the active ingredient in the formulation is between 20% and 50%. The percentage range of the active ingredient in the formulation is between 20% and 55%. The percentage range of the active ingredient in the formulation is between 20% and 60%. The percentage range of the active ingredient in the formulation is between 20% and 65%. The percentage range of the active ingredient in the formulation is between 20% and 70%. The percentage range of the active ingredient in the formulation is between 20% and 75%. The percentage range of the active ingredient in the formulation is between 20% and 80%. The percentage range of the active ingredient in the formulation is between 25% and 30%. The percentage range of the active ingredient in the formulation is between 25% and 35%. The percentage range of the active ingredient in the formulation is between 25% and 40%. The percentage range of the active ingredient in the formulation is between 25% and 45%. The percentage range of the active ingredient in the formulation is between 25% and 50%. The percentage range of the active ingredient in the formulation is between 25% and 55%. The percentage range of the active ingredient in the formulation is between 25% and 60%. The percentage range of the active ingredient in the formulation is between 25% and 65%. The percentage range of the active ingredient in the formulation is between 25% and 70%. The percentage range of the active ingredient in the formulation is between 25% and 75%. The percentage range of the active ingredient in the formulation is between 25% and 80%. The percentage range of the active ingredient in the formulation is between 30% and 35%. The percentage range of the active ingredient in the formulation is between 30% and 40%. The percentage range of the active ingredient in the formulation is between 30% and 45%. The percentage range of the active ingredient in the formulation is between 30% and 50%. The percentage range of the active ingredient in the formulation is between 30% and 55%. The percentage range of the active ingredient in the formulation is between 30% and 60%. The percentage range of the active ingredient in the formulation is between 30% and 65%. The percentage range of the active ingredient in the formulation is between 30% and 70%. The percentage range of the active ingredient in the formulation is between 30% and 75%. The percentage range of the active ingredient in the formulation is between 30% and 80%. The percentage range of the active ingredient in the formulation is between 35% and 40%. The percentage range of the active ingredient in the formulation is between 35% and 45%. The percentage range of the active ingredient in the formulation is between 35% and 50%. The percentage range of the active ingredient in the formulation is between 35% and 55%. The percentage range of the active ingredient in the formulation is between 35% and 60%. The percentage range of the active ingredient in the formulation is between 35% and 65%. The percentage range of the active ingredient in the formulation is between 35% and 70%. The percentage range of the active ingredient in the formulation is between 35% and 75%. The percentage range of the active ingredient in the formulation is between 35% and 80%. The percentage range of the active ingredient in the formulation is between 40% and 45%. The percentage range of the active ingredient in the formulation is between 40% and 50%. The percentage range of the active ingredient in the formulation is between 40% and 55%. The percentage range of the active ingredient in the formulation is between 40% and 60%. The percentage range of the active ingredient in the formulation is between 40% and 65%. The percentage range of the active ingredient in the formulation is between 40% and 70%. The percentage range of the active ingredient in the formulation is between 40% and 75%. The percentage range of the active ingredient in the formulation is between 40% and 80%. The percentage range of the active ingredient in the formulation is between 45% and 50%. The percentage range of the active ingredient in the formulation is between 45% and 55%. The percentage range of the active ingredient in the formulation is between 45% and 60%. The percentage range of the active ingredient in the formulation is between 45% and 65%. The percentage range of the active ingredient in the formulation is between 45% and 70%. The percentage range of the active ingredient in the formulation is between 45% and 75%. The percentage range of the active ingredient in the formulation is between 45% and 80%. The percentage range of the active ingredient in the formulation is between 50% and 55%. The percentage range of the active ingredient in the formulation is between 50% and 60%. The percentage range of the active ingredient in the formulation is between 50% and 65%. The percentage range of the active ingredient in the formulation is between 50% and 70%. The percentage range of the active ingredient in the formulation is between 50% and 75%. The percentage range of the active ingredient in the formulation is between 50% and 80%. The percentage range of the active ingredient in the formulation is between 55% and 60%. The percentage range of the active ingredient in the formulation is between 55% and 65%. The percentage range of the active ingredient in the formulation is between 55% and 70%. The percentage range of the active ingredient in the formulation is between 55% and 75%. The percentage range of the active ingredient in the formulation is between 55% and 80%. The percentage range of the active ingredient in the formulation is between 60% and 65%. The percentage range of the active ingredient in the formulation is between 60% and 70%. The percentage range of the active ingredient in the formulation is between 60% and 75%. The percentage range of the active ingredient in the formulation is between 60% and 80%. The percentage range of the active ingredient in the formulation is between 65% and 70%. The percentage range of the active ingredient in the formulation is between 65% and 75%. The percentage range of the active ingredient in the formulation is between 65% and 80%. The percentage range of the active ingredient in the formulation is between 70% and 75%. The percentage range of the active ingredient in the formulation is between 70% and 80%. The percentage range of the active ingredient in the formulation is between 75% and 80%.

In some embodiments, the percentage of the active ingredient in the formulation is a percentage between 50% and 75%. The percentage may be 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 75%, or 75%. The percentage of the active ingredient in the formulation may be 50%. The percentage of the active ingredient in the formulation may be 51%. The percentage of the active ingredient in the formulation may be 52%. The percentage of the active ingredient in the formulation may be 53%. The percentage of the active ingredient in the formulation may be 54%. The percentage of the active ingredient in the formulation may be 55%. The percentage of the active ingredient in the formulation may be 56%. The percentage of the active ingredient in the formulation may be 57%. The percentage of the active ingredient in the formulation may be 58%. The percentage of the active ingredient in the formulation may be 59%. The percentage of the active ingredient in the formulation may be 60%. The percentage of the active ingredient in the formulation may be 61%. The percentage of the active ingredient in the formulation may be 62%. The percentage of the active ingredient in the formulation may be 63%. The percentage of the active ingredient in the formulation may be 64%. The percentage of the active ingredient in the formulation may be 65%. The percentage of the active ingredient in the formulation may be 66%. The percentage of the active ingredient in the formulation may be 67%. The percentage of the active ingredient in the formulation may be 68%. The percentage of the active ingredient in the formulation may be 69%. The percentage of the active ingredient in the formulation may be 70%. The percentage of the active ingredient in the formulation may be 71%. The percentage of the active ingredient in the formulation may be 72%. The percentage of the active ingredient in the formulation may be 73%. The percentage of the active ingredient in the formulation may be 75%. The percentage of the active ingredient in the formulation may be 75%.

In some embodiments, if there is more than one chemical stimuli which together is the active ingredient in the formulation, the percentage of each chemical stimuli in the formulation may range independently between 1% and 80%. The percentage range of at least one chemical stimuli may be 1% and 5%. The percentage range of at least one chemical stimuli may be 1% and 10%. The percentage range of at least one chemical stimuli may be 1% and 15%. The percentage range of at least one chemical stimuli may be 1% and 20%. The percentage range of at least one chemical stimuli may be 1% and 25%. The percentage range of at least one chemical stimuli may be 1% and 30%. The percentage range of at least one chemical stimuli may be 1% and 35%. The percentage range of at least one chemical stimuli may be 1% and 40%. The percentage range of at least one chemical stimuli may be 1% and 45%. The percentage range of at least one chemical stimuli may be 1% and 50%. The percentage range of at least one chemical stimuli may be 1% and 55%. The percentage range of at least one chemical stimuli may be 1% and 60%. The percentage range of at least one chemical stimuli may be 1% and 65%. The percentage range of at least one chemical stimuli may be 1% and 70%. The percentage range of at least one chemical stimuli may be 1% and 75%. The percentage range of at least one chemical stimuli may be 1% and 80%. The percentage range of at least one chemical stimuli may be 5% and 10%. The percentage range of at least one chemical stimuli may be 5% and 15%. The percentage range of at least one chemical stimuli may be 5% and 20%. The percentage range of at least one chemical stimuli may be 5% and 25%. The percentage range of at least one chemical stimuli may be 5% and 30%. The percentage range of at least one chemical stimuli may be 5% and 35%. The percentage range of at least one chemical stimuli may be 5% and 40%. The percentage range of at least one chemical stimuli may be 5% and 45%. The percentage range of at least one chemical stimuli may be 5% and 50%. The percentage range of at least one chemical stimuli may be 5% and 55%. The percentage range of at least one chemical stimuli may be 5% and 60%. The percentage range of at least one chemical stimuli may be 5% and 65%. The percentage range of at least one chemical stimuli may be 5% and 70%. The percentage range of at least one chemical stimuli may be 5% and 75%. The percentage range of at least one chemical stimuli may be 5% and 80%. The percentage range of at least one chemical stimuli may be 10% and 15%. The percentage range of at least one chemical stimuli may be 10% and 20%. The percentage range of at least one chemical stimuli may be 10% and 25%. The percentage range of at least one chemical stimuli may be 10% and 30%. The percentage range of at least one chemical stimuli may be 10% and 35%. The percentage range of at least one chemical stimuli may be 10% and 40%. The percentage range of at least one chemical stimuli may be 10% and 45%. The percentage range of at least one chemical stimuli may be 10% and 50%. The percentage range of at least one chemical stimuli may be 10% and 55%. The percentage range of at least one chemical stimuli may be 10% and 60%. The percentage range of at least one chemical stimuli may be 10% and 65%. The percentage range of at least one chemical stimuli may be 10% and 70%. The percentage range of at least one chemical stimuli may be 10% and 75%. The percentage range of at least one chemical stimuli may be 10% and 80%. The percentage range of at least one chemical stimuli may be 15% and 20%. The percentage range of at least one chemical stimuli may be 15% and 25%. The percentage range of at least one chemical stimuli may be 15% and 30%. The percentage range of at least one chemical stimuli may be 15% and 35%. The percentage range of at least one chemical stimuli may be 15% and 40%. The percentage range of at least one chemical stimuli may be 15% and 45%. The percentage range of at least one chemical stimuli may be 15% and 50%. The percentage range of at least one chemical stimuli may be 15% and 55%. The percentage range of at least one chemical stimuli may be 15% and 60%. The percentage range of at least one chemical stimuli may be 15% and 65%. The percentage range of at least one chemical stimuli may be 15% and 70%. The percentage range of at least one chemical stimuli may be 15% and 75%. The percentage range of at least one chemical stimuli may be 15% and 80%. The percentage range of at least one chemical stimuli may be 20% and 25%. The percentage range of at least one chemical stimuli may be 20% and 30%. The percentage range of at least one chemical stimuli may be 20% and 35%. The percentage range of at least one chemical stimuli may be 20% and 40%. The percentage range of at least one chemical stimuli may be 20% and 45%. The percentage range of at least one chemical stimuli may be 20% and 50%. The percentage range of at least one chemical stimuli may be 20% and 55%. The percentage range of at least one chemical stimuli may be 20% and 60%. The percentage range of at least one chemical stimuli may be 20% and 65%. The percentage range of at least one chemical stimuli may be 20% and 70%. The percentage range of at least one chemical stimuli may be 20% and 75%. The percentage range of at least one chemical stimuli may be 20% and 80%. The percentage range of at least one chemical stimuli may be 25% and 30%. The percentage range of at least one chemical stimuli may be 25% and 35%. The percentage range of at least one chemical stimuli may be 25% and 40%. The percentage range of at least one chemical stimuli may be 25% and 45%. The percentage range of at least one chemical stimuli may be 25% and 50%. The percentage range of at least one chemical stimuli may be 25% and 55%. The percentage range of at least one chemical stimuli may be 25% and 60%. The percentage range of at least one chemical stimuli may be 25% and 65%. The percentage range of at least one chemical stimuli may be 25% and 70%. The percentage range of at least one chemical stimuli may be 25% and 75%. The percentage range of at least one chemical stimuli may be 25% and 80%. The percentage range of at least one chemical stimuli may be 30% and 35%. The percentage range of at least one chemical stimuli may be 30% and 40%. The percentage range of at least one chemical stimuli may be 30% and 45%. The percentage range of at least one chemical stimuli may be 30% and 50%. The percentage range of at least one chemical stimuli may be 30% and 55%. The percentage range of at least one chemical stimuli may be 30% and 60%. The percentage range of at least one chemical stimuli may be 30% and 65%. The percentage range of at least one chemical stimuli may be 30% and 70%. The percentage range of at least one chemical stimuli may be 30% and 75%. The percentage range of at least one chemical stimuli may be 30% and 80%. The percentage range of at least one chemical stimuli may be 35% and 40%. The percentage range of at least one chemical stimuli may be 35% and 45%. The percentage range of at least one chemical stimuli may be 35% and 50%. The percentage range of at least one chemical stimuli may be 35% and 55%. The percentage range of at least one chemical stimuli may be 35% and 60%. The percentage range of at least one chemical stimuli may be 35% and 65%. The percentage range of at least one chemical stimuli may be 35% and 70%. The percentage range of at least one chemical stimuli may be 35% and 75%. The percentage range of at least one chemical stimuli may be 35% and 80%. The percentage range of at least one chemical stimuli may be 40% and 45%. The percentage range of at least one chemical stimuli may be 40% and 50%. The percentage range of at least one chemical stimuli may be 40% and 55%. The percentage range of at least one chemical stimuli may be 40% and 60%. The percentage range of at least one chemical stimuli may be 40% and 65%. The percentage range of at least one chemical stimuli may be 40% and 70%. The percentage range of at least one chemical stimuli may be 40% and 75%. The percentage range of at least one chemical stimuli may be 40% and 80%. The percentage range of at least one chemical stimuli may be 45% and 50%. The percentage range of at least one chemical stimuli may be 45% and 55%. The percentage range of at least one chemical stimuli may be 45% and 60%. The percentage range of at least one chemical stimuli may be 45% and 65%. The percentage range of at least one chemical stimuli may be 45% and 70%. The percentage range of at least one chemical stimuli may be 45% and 75%. The percentage range of at least one chemical stimuli may be 45% and 80%. The percentage range of at least one chemical stimuli may be 50% and 55%. The percentage range of at least one chemical stimuli may be 50% and 60%. The percentage range of at least one chemical stimuli may be 50% and 65%. The percentage range of at least one chemical stimuli may be 50% and 70%. The percentage range of at least one chemical stimuli may be 50% and 75%. The percentage range of at least one chemical stimuli may be 50% and 80%. The percentage range of at least one chemical stimuli may be 55% and 60%. The percentage range of at least one chemical stimuli may be 55% and 65%. The percentage range of at least one chemical stimuli may be 55% and 70%. The percentage range of at least one chemical stimuli may be 55% and 75%. The percentage range of at least one chemical stimuli may be 55% and 80%. The percentage range of at least one chemical stimuli may be 60% and 65%. The percentage range of at least one chemical stimuli may be 60% and 70%. The percentage range of at least one chemical stimuli may be 60% and 75%. The percentage range of at least one chemical stimuli may be 60% and 80%. The percentage range of at least one chemical stimuli may be 65% and 70%. The percentage range of at least one chemical stimuli may be 65% and 75%. The percentage range of at least one chemical stimuli may be 65% and 80%. The percentage range of at least one chemical stimuli may be 70% and 75%. The percentage range of at least one chemical stimuli may be 70% and 80%. The percentage range of at least one chemical stimuli may be 75% and 80%.

In some embodiments, if there is more than one chemical stimuli in the formulation, the percentage of each chemical stimuli in the formulation may independently be between 1% and 80%. The percentage of at least one chemical stimuli may be 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, or 75%. The percentage of at least one chemical stimuli may be 1%. The percentage of at least one chemical stimuli may be 2%. The percentage of at least one chemical stimuli may be 3%. The percentage of at least one chemical stimuli may be 4%. The percentage of at least one chemical stimuli may be 5%. The percentage of at least one chemical stimuli may be 6%. The percentage of at least one chemical stimuli may be 7%. The percentage of at least one chemical stimuli may be 8%. The percentage of at least one chemical stimuli may be 9%. The percentage of at least one chemical stimuli may be 10%. The percentage of at least one chemical stimuli may be 11%. The percentage of at least one chemical stimuli may be 12%. The percentage of at least one chemical stimuli may be 13%. The percentage of at least one chemical stimuli may be 14%. The percentage of at least one chemical stimuli may be 15%. The percentage of at least one chemical stimuli may be 16%. The percentage of at least one chemical stimuli may be 17%. The percentage of at least one chemical stimuli may be 18%. The percentage of at least one chemical stimuli may be 19%. The percentage of at least one chemical stimuli may be 20%. The percentage of at least one chemical stimuli may be 25%. The percentage of at least one chemical stimuli may be 30%. The percentage of at least one chemical stimuli may be 35%. The percentage of at least one chemical stimuli may be 40%. The percentage of at least one chemical stimuli may be 45%. The percentage of at least one chemical stimuli may be 50%. The percentage of at least one chemical stimuli may be 55%. The percentage of at least one chemical stimuli may be 60%. The percentage of at least one chemical stimuli may be 65%. The percentage of at least one chemical stimuli may be 70%. The percentage of at least one chemical stimuli may be 75%. The percentage of at least one chemical stimuli may be 80%.

### Exemplary Active Ingredients

In some embodiments, the formulation includes at least two chemical stimuli as active ingredients. The percentage of chemical stimuli may be independently selected from a percentage such as, but not limited to, 25%, 33%, 33.3%, 50%, 51%, 52%, 53%, 54%, 55%, 56%, 57%, 58%, 59%, 60%, 61%, 62%, 63%, 64%, 65%, 66%, 66.7%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or more than 90%. The percentage range of the chemical stimuli may be independently selected from 25% to 30%, 25% to 35%, 25% to 40%, 25% to 45%, 25% to 50%, 25% to 55%, 25% to 60%, 25% to 65%, 25% to 70%, 25% to 75%, 30% to 35%, 30% to 40%, 30% to 45%, 30% to 50%, 30% to 55%, 30% to 60%, 30% to 65%, 30% to 70%, 30% to 75%, 35% to 40%, 35% to 45%, 35% to 50%, 35% to 55%, 35% to 60%, 35% to 65%, 35% to 70%, 35% to 75%, 40% to 45%, 40% to 50%, 40% to 55%, 40% to 60%, 40% to 65%, 40% to 70%, 40% to 75%, 45% to 50%, 45% to 55%, 45% to 60%, 45% to 65%, 45% to 70%, 45% to 75%, 50% to 55%, 50% to 60%, 50% to 65%, 50% to 70%, 50% to 75%, 55% to 60%, 55% to 65%, 55% to 70%, 55% to 75%, 60% to 65%, 60% to 70%, 60% to 75%, 65% to 70%, 65% to 75%, and 70% to 75%.

In some embodiments, the formulation includes two chemical stimuli as active ingredients. The percentage of chemical stimuli may be independently selected from a percentage such as, but not limited to, 25%, 33%, 33.3%, 50%, 51%, 52%, 53%, 54%, 55%, 56%, 57%, 58%, 59%, 60%, 61%, 62%, 63%, 64%, 65%, 66%, 66.7%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or more than 90%.

In some embodiments, the formulations include Malic acid (e.g., D-Malic acid, L-Malic acid, D/L-Malic acid) and mannitol as the active ingredients. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 66.7% and the percentage of the mannitol is 33.3%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 25% and the percentage of the mannitol is 75%.

In some embodiments, the formulations include L-Malic acid and mannitol as the active ingredients. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%.

In some embodiments, the formulations include D-Malic acid and mannitol as the active ingredients. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%.

In some embodiments, the formulations include D/L-Malic acid and mannitol as the active ingredients. As a non-limiting example, the percentage of the active ingredient which is 25% and the other 75% is D/L-Malic acid. As a non-limiting example, the percentage of the active ingredient which is 33.3% and the other 67.5% is D/L-Malic acid. As a non-limiting example, the percentage of the active ingredient which is 50% and the other 50% is D/L-Malic acid. As a non-limiting example, the percentage of the active ingredient which is 75% and the other 25% is D/L-Malic acid.

The formulations described herein may include Malic acid (e.g., D-Malic acid, L-Malic acid, D/L-Malic acid) and at least one bulking agent, filler or disintegrant. As a non-limiting example, the percentage of the Malic acid (e.g., D-Malic acid, L-Malic acid, D/L-Malic acid) is 75% and the percentage of the bulking agent, filler or disintegrant is 25%. As a non-limiting example, the percentage of the Malic acid (e.g., D-Malic acid, L-Malic acid, D/L-Malic acid) is 66.7% and the percentage of the bulking agent, filler or disintegrant is 33.3%. As a non-limiting example, the percentage of the Malic acid (e.g., D-Malic acid, L-Malic acid, D/L-Malic acid) is 50% and the percentage of the bulking agent, filler or disintegrant is 50%. As a non-limiting example, the percentage of the Malic acid (e.g., D-Malic acid, L-Malic acid, D/L-Malic acid) is 25% and the percentage of the bulking agent, filler or disintegrant is 75%.

### Excipients

The formulation described herein may include at least one excipient. In some embodiments, the formulation may be a pharmaceutical composition. Excipients may include, but are not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components.

The formulations and/or pharmaceutical compositions described herein may include at least one excipient which is a sugar or sugar alcohol. Non-limiting examples of sugar or sugar alcohols include dextrose, dextrin, glucose, sucrose, isomaltitol, lactitol, lactose, mannitol, sorbitol, sucrose, and xylitol.

In some embodiments, an excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The formulations and/or pharmaceutical compositions described herein may include at least one excipient which is a lubricant. Non-limiting examples of lubricants are talc, sodium lauryl sulfate, solid polyethylene glycols, silicone substances such as silicone dioxide, and stearates such as calcium stearate, magnesium stearate, and sodium stearate. The concentration of the lubricant in the formulation and/or pharmaceutical composition may be less than 1% to up to 5% such as, but not limited to, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, and 5.0% or 0.1% to 0.5%, 0.1% to 1%, 0.1% to 2%, 0.1% to 3%, 0.1% to 4%, 0.1% to 5%, 0.5% to 1%, 0.5% to 2%, 0.5% to 3%, 0.5% to 4%, 0.5% to 5%, 1.0% to 2%, 1.0% to 3%, 1.0% to 4%, 1.0% to 5%, 1.5% to 2%, 1.5% to 3%, 1.5% to 4%, 1.5% to 5%, 2.0% to 3%, 2.0% to 4%, 2.0% to 5%, 2.5% to 3%, 2.5% to 4%, 2.5% to 5%, 3% to 4%, 3% to 5%, 3.5% to 4%, 3.5% to 5%, 4% to 5%, and 4.5% to 5%.

The formulations and/or pharmaceutical compositions described herein may include at least one excipient which is a disintegrant. Non-limiting examples of disintegrants are crospovidone (xPVP), sodium croscarmellose (CCS), sodium bicarbonate, magnesium bicarbonate, starch (e.g., pregelatinized starch, modified starch such as sodium starch glyycolate), cellulose and its derivatives (e.g., sodium carboxy methylcellulose, microcystalline cellulose (MCC)), and alginates (e.g., alginic acid and sodium alginate). The concentration of the disintegrant in the formulation and/or pharmaceutical composition may be less than 1% to up to 20% such as, but not limited to, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%. The concentration range of the disintegrant in the formulation and/or pharmaceutical composition may be 1% to 20%, 1% to 15%, 1% to 10%, 1% to 9%, 1% to 8%, 1% to 7%, 1% to 6%, 1% to 5%, 1% to 4%, 1% to 3%, 1% to 2%, 2% to 20%, 2% to 15%, 2% to 10%, 2% to 9%, 2% to 8%, 2% to 7%, 2% to 6%, 2% to 5%, 2% to 4%, 2% to 3%, 3% to 20%, 3% to 15%, 3% to 10%, 3% to 9%, 3% to 8%, 3% to 7%, 3% to 6%, 3% to 5%, 3% to 4%, 4% to 20%, 4% to 15%, 4% to 10%, 4% to 9%, 4% to 8%, 4% to 7%, 4% to 6%, 4% to 5%, 5% to 20%, 5% to 15%, 5% to 10%, 5% to 9%, 5% to 8%, 5% to 7%, 5% to 6%, 6% to 20%, 6% to 15%, 6% to 10%, 6% to 9%, 6% to 8%, 6% to 7%, 7% to 20%, 7% to 15%, 7% to 10%, 7% to 9%, 7% to 8%, 8% to 20%, 8% to 15%, 8% to 10%, 8% to 9%, 9% to 20%, 9% to 15%, 9% to 10%, 10% to 20%, 10% to 15%, or 15% to 20%. As a non-limiting example, the concentration range of the disintegrant in the formulation and/or pharmaceutical composition is 1% to 20%.As a non-limiting example, the concentration range of the disintegrant in the formulation and/or pharmaceutical composition is 4% to 8%. Pharmaceutical Compositions

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g., chemical stimuli), the excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

### Administration and Delivery

In some embodiments, the active ingredient(s) may be formulated as a solid dosage form. The solid dosage form may be formed by any method known in the art and/or described herein. Non-limiting examples of solid dosage forms are orally disintegrating tablets (ODTs). ODTs may be made by methods such as, but not limited to, direct compression, freeze drying or lyophilization, spray drying, molding, phase transition process, melt granulation, sublimation, mass extrusion, cotton candy process, disintegrant addition, and nanonization.

In some embodiments, the active ingredient(s) may be formulated for oral delivery.

### Oral Administration

In some embodiments, the active ingredients may be formulated to be administered orally. Solid dosage forms for oral administration include, but are not limited to, capsules, tablets (including chewable tablets and chewable tablets), pills (including chewable pills and dissolvable pills), powders, gels (including dissolvable gels) and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

In some embodiments, the active ingredients may be formulated to be administered orally with a tablet that disintegrates upon contact with a liquid (e.g., saliva). This may be referred to as an orally disintegrating tablet (ODT).

In some embodiments, the active ingredients may be formulated to be administered orally as a capsule.

In some embodiments, the active ingredients may be formulated to be administered orally as a tablet.

In some embodiments, the active ingredients may be formulated to be administered orally as a chewable tablet.

In some embodiments, the active ingredients may be formulated to be administered orally as a hard candy.

In some embodiments, the active ingredients may be formulated to be administered orally as a hard candy such as, but not limited to, a lollipop.

In some embodiments, the active ingredients may be formulated to be administered orally as a powder.

In some embodiments, the oral formulation is a powder that is to be dissolved in a fluid to form a solution for drinking.

In some embodiments, the oral formulation is a powder with an effervescent that is to be dissolved in a fluid to form a solution for drinking. As a non-limiting example, the oral formulation includes an acid as the active ingredient and a base as an excipient to create an effervescent formulation which mixed with a fluid.

In some embodiments, the oral formulation is a tablet with an effervescent that is to be dissolved in a fluid to form a solution for drinking. As a non-limiting example, the oral formulation includes an acid as the active ingredient and a base as an excipient to create an effervescent formulation which mixed with a fluid.

In some embodiments, the active ingredients may be formulated to be administered orally as a soft, cohesive substance designed to be chewed without being swallowed (e.g., gum).

In some embodiments, the active ingredients may be formulated to be administered orally as a thin film. The film may disintegrate over a period of time (e.g., disintegrate after 1 hour, 2 hours, 3 hours, 4 hours, 5 hours, 6 hours or more than 6 hours) after contact with a water-based solution (e.g., saliva) or the film may not be soluble in water.

In some embodiments, the active ingredients may be formulated for oral administration to be administered to the oral mucosa.

In some embodiments, the active ingredients may be formulated for oral administration to be administered sublingually.

In some embodiments, the active ingredients may be formulated for oral administration to be administered to the soft palate.

In some embodiments, the active ingredients may be formulated for oral administration to be administered to the tongue.

Parenteral administration

In some embodiments, the active ingredients may be formulated to be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropyl cellulose.

In order to prolong the effect of active ingredients, it is often desirable to slow the absorption of active ingredients from parenteral administration. Delayed absorption of a parenterally administered drug form is accomplished by dissolving or suspending the drug in an oil vehicle.

In some embodiments, the active ingredients may be formulated for parenteral administration to be administered to the oral mucosa.

In some embodiments, the active ingredients may be formulated for parenteral administration to be administered sublingually.

In some embodiments, the active ingredients may be formulated for parenteral administration to be administered to the soft palate.

In some embodiments, the active ingredients may be formulated for parenteral administration to be administered to the tongue. Injectable administration

In some embodiments, the active ingredients may be formulated to be administered by injection. As a non-limiting example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

In some embodiments, the active ingredients may be formulated for injectable administration to be administered to the oral mucosa.

In some embodiments, the active ingredients may be formulated for injectable administration to be administered sublingually.

In some embodiments, the active ingredients may be formulated for injectable administration to be administered to the soft palate.

In some embodiments, the active ingredients may be formulated for injectable administration to be administered to the tongue.

### Topical or Transdermal Administration

In some embodiments, the active ingredients may be formulated to be administered topically. The skin may be an ideal target site for delivery as it is readily accessible. Three routes are commonly considered to deliver formulations of the active ingredients described herein to the skin: (i) topical application (e.g. for local/regional treatment); (ii) intradermal injection (e.g. for local/regional treatment); and (iii) systemic delivery (e.g. for treatment of dermatologic diseases that affect both cutaneous and extracutaneous regions). Formulations of the active ingredients described herein can be delivered to the skin by several different approaches known in the art.

In some embodiments, the route of administration may be through use of a variety of dressings (e.g., wound dressings) or bandages (e.g., adhesive bandages).

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of the formulations of active agents to the body. Such dosage forms may be prepared, for example, by dissolving and/or dispensing formulations of active agents in the proper medium. Alternatively, or additionally, rates may be controlled by either providing rate controlling membranes and/or by dispersing formulations of active agents in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

In some embodiments, the active ingredients may be formulated for topical or transdermal administration to be administered to the oral mucosa.

In some embodiments, the active ingredients may be formulated for topical or transdermal administration to be administered sublingually.

In some embodiments, the active ingredients may be formulated for topical or transdermal administration to be administered to the soft palate.

In some embodiments, the active ingredients may be formulated for topical or transdermal administration to be administered to the tongue.

### Depot Administration

As described herein, in some embodiments, active agents described herein are formulated in depots for extended release. Generally, specific organs or tissues (“target tissues”) are targeted for administration.

In some embodiments, formulations of active agents are spatially retained within or proximal to target tissues. Provided are methods of providing formulations of active agents to target tissues of mammalian subjects by contacting target tissues (which comprise one or more target cells) with formulations of active agents, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the formulation is retained in the target tissues. Intranasal, nasal and buccal administration

In some embodiments, formulations of active agents may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, e.g., by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### IV. Therapeutic Uses: Respiratory Arrythmia

In some embodiment, the formulations of active ingredients described herein may be used to treat respiratory arrythmia. As a non-limiting example, the respiratory arrythmia is chronic hiccups. As a non-limiting example, the respiratory arrythmia is sporadic hiccups. As a non-limiting example, the respiratory arrythmia is “idiopathic hiccups” which are hiccups which arise spontaneously or for which the cause is unknown. As a non-limiting example, the respiratory arrythmia is idiopathic hiccups which may be chronic hiccups. As a non-limiting example, the respiratory arrythmia is idiopathic hiccups which may be sporadic hiccups.

### Chronic Hiccups

One example of a condition which is associated with respiratory arrhythmia is “chronic hiccups” where there several cycles of normal respiratory rhythm (eupnea) separated by at least one period of rapid and high amplitude inspirations (hiccups) which last for an extended period of time longer than 24 hours. The period of time may be, but is not limited to more than 24 hours, 28 hours, 32 hours, 36 hours, 40 hours, 44 hours, 48 hours, 52 hours, 56 hours, 60 hours, 64 hours, 68 hours, 72 hours, 76 hours, 80 hours, 84 hours, 88 hours, 92 hours, 96 hours, 100 hours, 104 hours, 108 hours, 112 hours, 116 hours, 120 hours, 124 hours, 128 hours, 132 hours, 136 hours, 140 hours, 144 hours, 148 hours, 152 hours, 156 hours, 160 hours, 164 hours, 168 hours, 172 hours, 176 hours, 180 hours, 184 hours, 188 hours, 192 hours, 196 hours, 200 hours, 204 hours, 208 hours, 212 hours, 216 hours, 220 hours, 224 hours, 228 hours, 232 hours, 236 hours, 240 hours, 244 hours, 248 hours, 252 hours, 256 hours, 260 hours, 264 hours, 268 hours, 272 hours, 276 hours, 280 hours, 284 hours, 288 hours, 292 hours, 296 hours, 300 hours, 304 hours, 308 hours, 312 hours, 316 hours, 320 hours, 324 hours, 328 hours, 332 hours, or more than 336 hours. The term “chronic hiccup” may be used interchangeably with “intractable hiccups” or “persistent hiccups.”

Chronic hiccups have been a side effect of various diseases, disorders and/or conditions such as, but not limited to, cancer, advanced cancer, brain injury, neuromyelitis optica (NMO), Parkinson's disease, multiple sclerosis (MS), epilepsy, stroke, traumatic brain injury, dementia, Alzheimer's disease, Hick's disease, Huntington's disease, Chemotherapy-induced Nausea and Vomiting (CINV), gastroesophageal reflux disease (GERD), Kidney Failure (ESRD), anesthesia, surgery, and cardiovascular-related disease.

Chronic hiccups may also be a side effect of medication. In some embodiments, chronic hiccups are caused by large amounts of medication. In some embodiments, chronic hiccups are the result of chemotherapy. In some embodiments, chronic hiccups are the result of anesthesia.

If not treated, complications of chronic hiccups include, but are not limited to, anxiety, aspiration syndromes, bradycardia/heart block, wound dehiscence, weight loss, vomiting, dehydration, depression, dyspnea/hypoxia, subcutaneous emphysema, speech impairment, fatigue, malnutrition, and pneumomediastinum.

There have been different treatments that have been explored to treat chronic hiccups but these treatments have either not been successful or not practical for ease of use. Previously explored treatments include digital rectal massage, sexual intercourse, and increasing inhaled carbon dioxide levels.

The present disclosure provides compositions and methods of treatment for chronic hiccups.

The present disclosure provides compositions and methods of treatment for chronic hiccups to a subject who is or has been an oncology patient.

In some embodiments, the formulations of active ingredients described herein may be used to treat and/or prevent chronic hiccups. In some embodiments, the effect of chronic hiccups is reduced so a subject does not hiccup for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours (5 days) or more than 120 hours ( 5 days).

In some embodiments, to treat and/or prevent chronic hiccups, the present formulation which includes Malic acid (e.g., D-Malic acid, L-Malic acid, D/L-Malic acid) and/or mannitol as the active ingredient may be administered or delivered to a subject. The active ingredient may be made of 25% to 75% of D-Malic acid, L-Malic acid or D/L-Malic acid. Additionally, the active ingredient may also include 25% to 75% of mannitol. The mannitol may be present in the D or L form or a combination thereof.

In some embodiments, to treat and/or prevent chronic hiccups, the present formulation which includes Malic acid (e.g., D-Malic acid, L-Malic acid, D/L-Malic acid) and mannitol as the active ingredients may be administered or delivered to a subject. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 66.7% and the percentage of the mannitol is 33.3%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 25% and the percentage of the mannitol is 75%.

In some embodiments, to treat and/or prevent chronic hiccups, the present formulation which includes L-Malic acid and mannitol as the active ingredients may be administered or delivered to a subject. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%.

In some embodiments, to treat and/or prevent chronic hiccups, the present formulation which includes D-Malic acid and mannitol as the active ingredients may be administered or delivered to a subject. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%.

In some embodiments, to treat and/or prevent chronic hiccups, the present formulation which includes D/L-Malic acid and mannitol as the active ingredients may be administered or delivered to a subject. As a non-limiting example, the percentage of the active ingredient which is mannitol is 25% and the other 75% is D/L-Malic acid. As a non-limiting example, the percentage of the active ingredient which is mannitol is 50% and the other 50% is D/L-Malic acid. As a non-limiting example, the percentage of the active ingredient which is mannitol is 33.3% and the other 67.5% is D/L-Malic acid. As a non-limiting example, the percentage of the active ingredient which is 75% and the other 25% is a D/L-Malic acid.

In some embodiment, the compositions described herein may be used in combination with other perturbations described herein.

In some embodiment, the compositions described herein may be used in combination with other perturbations which are known to treat hiccups. Non-limiting examples of perturbations include antagonists such as a 5-HT3 receptor antagonist, a dopamine antagonist, or an NK1 receptor antagonist, or nerve blockers or stimulations.

In some embodiments, provided are pharmaceutical compositions to treat respiratory arrythmia (e.g., chronic hiccups) comprising (a) one or more perturbation stimuli selected from chemical stimuli, external electrical stimuli, and external mechanical stimuli and (b) one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol.

In some embodiment, provided are orally disintegrating tablets (ODT) to treat respiratory arrythmia (e.g., chronic hiccups) comprising one or more perturbation stimuli selected from chemical stimuli, external electrical stimuli, and external mechanical stimuli and one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol.

In some embodiments, provided are orally disintegrating tablets (ODT) to treat respiratory arrythmia (e.g., chronic hiccups) comprising one or more chemical stimuli and sugar or sugar alcohols. As a non-limiting example, the ODT may include about 40-80% of the at least one chemical stimuli and about 20-60% of the sugar or sugar alcohols. As a non-limiting example, the ODT comprises about 50% of the at least one chemical stimuli and about 50% of the sugar or sugar alcohols. As a non-limiting example, the ODT comprises about 75% of the at least one chemical stimuli and about 25% of the sugar or sugar alcohols.

Provided herein are methods of treating a disease, disorder or condition related to respiratory arrythmia comprising administering a therapeutically effective amount of the pharmaceutical composition or the ODT described in the preceding paragraphs to a subject in need thereof. The respiratory arrythmia may be caused by a disease, disorder or condition selected from the group consisting of cancer, advanced cancer, apnea, orthopnea, dyspnea, hyperpnea, hyperventilation, hypoventilation, tachypnea, Kussmaul respiration, Cheyne-Stokes respiration, sighing respiration, Biot respiration, apneustic breathing, central neurogenic hyperventilation, central neurogenic hypoventilation, agonal breathing, allergies, multiple sclerosis, Parkinson disease, Trauma, Postpolio syndrome, Amyotrophic Lateral Sclerosis, Guillain-Barré syndrome, Charcot-Marie-Tooth disease, Myasthenia gravis, Botulism, Duchenne muscular dystrophy, Polymyositis/dermatomyositis, Postparalysis myopathy, asthma, chronic obstructive pulmonary disease (COPD) and interstitial lung disease, anxiety related disorders, gastroesophageal reflux disease (GERD), panic disorder, Rett syndrome, effects of post-surgery recovery, weight loss, and insomnia. As a non-limiting example, the disease, disorder or condition related to respiratory arrythmia is chronic hiccups. In some aspects, the pharmaceutical composition or ODT reduces the effect of chronic hiccups so a subject does not hiccup for a times selected from the group consisting of 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours (5 days) and more than 120 hours ( 5 days).

### Sporadic Hiccups

One example of a condition which is associated with respiratory arrhythmia is “sporadic hiccups” where there are irregular interval cycles of normal respiratory rhythm (eupnea) separated by at least one period of rapid and high amplitude inspirations (hiccups). These sporadic hiccups can last for an interval from a few minutes (e.g., 1, 2, 3, 4, 5, 6, 7, 8, 9, 10, 15, 20, 25, 30, 35, 40, 45, 50, 55) to a few hours (1, 2, 3, 4, 5, 6, 7, 8, 9, 10, 11, 12, 13, 14, 15, 16, 17, 18, 19, 20, 21, 22, 23) but not longer than 24 hours. The interval cycle is irregular and may repeat randomly over a period of time longer than 24 hours. The period of time may be, but is not limited to more than 24 hours, 28 hours, 32 hours, 36 hours, 40 hours, 44 hours, 48 hours, 52 hours, 56 hours, 60 hours, 64 hours, 68 hours, 72 hours, 76 hours, 80 hours, 84 hours, 88 hours, 92 hours, 96 hours, 100 hours, 104 hours, 108 hours, 112 hours, 116 hours, 120 hours, 124 hours, 128 hours, 132 hours, 136 hours, 140 hours, 144 hours, 148 hours, 152 hours, 156 hours, 160 hours, 164 hours, 168 hours, 172 hours, 176 hours, 180 hours, 184 hours, 188 hours, 192 hours, 196 hours, 200 hours, 204 hours, 208 hours, 212 hours, 216 hours, 220 hours, 224 hours, 228 hours, 232 hours, 236 hours, 240 hours, 244 hours, 248 hours, 252 hours, 256 hours, 260 hours, 264 hours, 268 hours, 272 hours, 276 hours, 280 hours, 284 hours, 288 hours, 292 hours, 296 hours, 300 hours, 304 hours, 308 hours, 312 hours, 316 hours, 320 hours, 324 hours, 328 hours, 332 hours, or more than 336 hours. The term “sporadic hiccup” may be used interchangeably with “acute hiccups.”

Sporadic hiccups have been a side effect of various diseases, disorders and/or conditions such as, but not limited to, cancer, advanced cancer, brain injury, neuromyelitis optica (NMO), Parkinson's disease, multiple sclerosis (MS), epilepsy, stroke, traumatic brain injury, dementia, Alzheimer's disease, Hick's disease, Huntington's disease, Chemotherapy-induced Nausea and Vomiting (CINV), gastroesophageal reflux disease (GERD), Kidney Failure (ESRD), anesthesia, surgery, and cardiovascular-related disease.

Sporadic hiccups may also be a side effect of medication. In some embodiments, sporadic hiccups are caused by large amounts of medication. In some embodiments, sporadic hiccups are the result of chemotherapy. In some embodiments, sporadic hiccups are the result of anesthesia.

If not treated, complications of sporadic hiccups include, but are not limited to, anxiety, aspiration syndromes, bradycardia/heart block, wound dehiscence, weight loss, vomiting, dehydration, depression, dyspnea/hypoxia, subcutaneous emphysema, speech impairment, fatigue, malnutrition, and pneumomediastinum.

There have been different treatments that have been explored to treat sporadic hiccups but these treatments have either not been successful or not practical for ease of use. Previously explored treatments include digital rectal massage, sexual intercourse, and increasing inhaled carbon dioxide levels.

The present disclosure provides compositions and methods of treatment for sporadic hiccups.

The present disclosure provides compositions and methods of treatment for sporadic hiccups to a subject who is or has been an oncology patient.

In some embodiments, the formulations of active ingredients described herein may be used to treat and/or prevent sporadic hiccups. In some embodiments, the effect of sporadic hiccups is reduced so a subject does not hiccup for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), hours, 48 hours ( 2 days), 60 hours, 72 hours ( 3 days), 84 hours, 96 hours ( 4 days), 108 hours, 120 hours ( 5 days) or more than 120 hours ( 5 days).

In some embodiments, to treat and/or prevent sporadic hiccups, the present formulation which includes Malic acid (e.g., D-Malic acid, L-Malic acid, D/L-Malic acid) and/or mannitol as the active ingredient may be administered or delivered to a subject. The active ingredient may be made of 25% to 75% of D-Malic acid, L-Malic acid or D/L-Malic acid. Additionally, the active ingredient may also include 25% to 75% of mannitol. The mannitol may be present in the D or L form or a combination thereof.

In some embodiments, to treat and/or prevent sporadic hiccups, the present formulation which includes Malic acid (e.g., D-Malic acid, L-Malic acid, D/L-Malic acid) and mannitol as the active ingredients may be administered or delivered to a subject. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 66.7% and the percentage of the mannitol is 33.3%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 25% and the percentage of the mannitol is 75%.

In some embodiments, to treat and/or prevent sporadic hiccups, the present formulation which includes L-Malic acid and mannitol as the active ingredients may be administered or delivered to a subject. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%.

In some embodiments, to treat and/or prevent sporadic hiccups, the present formulation which includes D-Malic acid and mannitol as the active ingredients may be administered or delivered to a subject. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%.

In some embodiments, to treat and/or prevent sporadic hiccups, the present formulation which includes D/L-Malic acid and mannitol as the active ingredients may be administered or delivered to a subject. As a non-limiting example, the percentage of the active ingredient which is mannitol is 25% and the other 75% is D/L-Malic acid. As a non-limiting example, the percentage of the active ingredient which is mannitol is 50% and the other 50% is D/L-Malic acid. As a non-limiting example, the percentage of the active ingredient which is mannitol is 33.3% and the other 67.5% is D/L-Malic acid. As a non-limiting example, the percentage of the active ingredient which is 75% and the other 25% is a D/L-Malic acid.

In some embodiment, the compositions described herein may be used in combination with other perturbations described herein.

In some embodiment, the compositions described herein may be used in combination with other perturbations which are known to treat hiccups. Non-limiting examples of perturbations include antagonists such as a 5-HT3 receptor antagonist, a dopamine antagonist, or an NK1 receptor antagonist, or nerve blockers or stimulations.

In some embodiments, provided are pharmaceutical compositions to treat respiratory arrythmia (e.g., sporadic hiccups) comprising (a) one or more perturbation stimuli selected from chemical stimuli, external electrical stimuli, and external mechanical stimuli and (b) one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol.

In some embodiment, provided are orally disintegrating tablets (ODT) to treat respiratory arrythmia (e.g., sporadic hiccups) comprising one or more perturbation stimuli selected from chemical stimuli, external electrical stimuli, and external mechanical stimuli and one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol.

In some embodiments, provided are orally disintegrating tablets (ODT) to treat respiratory arrythmia (e.g., sporadic hiccups) comprising one or more chemical stimuli and sugar or sugar alcohols. As a non-limiting example, the ODT may include about 40-80% of the at least one chemical stimuli and about 20-60% of the sugar or sugar alcohols. As a non-limiting example, the ODT comprises about 50% of the at least one chemical stimuli and about 50% of the sugar or sugar alcohols. As a non-limiting example, the ODT comprises about 75% of the at least one chemical stimuli and about 25% of the sugar or sugar alcohols.

Provided herein are methods of treating a disease, disorder or condition related to respiratory arrythmia comprising administering a therapeutically effective amount of the pharmaceutical composition or the ODT described in the preceding paragraphs to a subject in need thereof. The respiratory arrythmia may be caused by a disease, disorder or condition selected from the group consisting of cancer, advanced cancer, apnea, orthopnea, dyspnea, hyperpnea, hyperventilation, hypoventilation, tachypnea, Kussmaul respiration, Cheyne-Stokes respiration, sighing respiration, Biot respiration, apneustic breathing, central neurogenic hyperventilation, central neurogenic hypoventilation, agonal breathing, allergies, multiple sclerosis, Parkinson disease, Trauma, Postpolio syndrome, Amyotrophic Lateral Sclerosis, Guillain-Barré syndrome, Charcot-Marie-Tooth disease, Myasthenia gravis, Botulism, Duchenne muscular dystrophy, Polymyositis/dermatomyositis, Postparalysis myopathy, asthma, sporadic obstructive pulmonary disease (COPD) and interstitial lung disease, anxiety related disorders, gastroesophageal reflux disease (GERD), panic disorder, Rett syndrome, effects of post-surgery recovery, weight loss, and insomnia. As a non-limiting example, the disease, disorder or condition related to respiratory arrythmia is sporadic hiccups. In some aspects, the pharmaceutical composition or ODT reduces the effect of sporadic hiccups so a subject does not hiccup for a times selected from the group consisting of 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) and more than 120 hours ( 5 days).

### V. Therapeutic Uses: Nausea and Vomiting

In some embodiment, the formulations of active ingredients described herein may be used to treat nausea.

In some embodiment, the formulations of active ingredients described herein may be used to treat vomiting.

### Nausea and Vomiting

In some embodiments, the formulations of active ingredients described herein may be used to treat and/or prevent nausea and/or vomiting. The nausea and/or vomiting may be due to a bacterial or virus infection or as a side-effect of medicine or medical therapy. In some embodiments, the effects of nausea and/or vomiting is reduced so a subject does not vomit for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours (3 days), 84 hours, 96 hours ( 4 days), 108 hours, 120 hours ( 5 days) or more than 120 hours (5 days).

In some embodiments, the formulations of active ingredients described herein may be used to treat and/or prevent Chemotherapy-induced Nausea and Vomiting (CINV). In some embodiments, the effects of CINV is reduced so a subject does not vomit for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) or more than 120 hours ( 5 days).

In some embodiments, the formulations of active ingredients described herein may be used in combination with chemotherapy agents.

In some embodiments, the formulations of active ingredients described herein may be used in combination with antiemetic drugs.

In some embodiments, to treat and/or prevent nausea and/or vomiting, the present formulation which includes Malic acid (e.g., D-Malic acid, L-Malic acid, D/L-Malic acid) and/or mannitol as the active ingredient may be administered or delivered to a subject. The active ingredient may be made of 25% to 75% of D-Malic acid, L-Malic acid or D/L-Malic acid. Additionally, the active ingredient may also include 25% to 75% of mannitol. The mannitol may be present in the D or L form or a combination thereof.

In some embodiments, to treat and/or prevent nausea and/or vomiting, the present formulation which includes Malic acid (e.g., D-Malic acid, L-Malic acid, D/L-Malic acid) and mannitol as the active ingredients may be administered or delivered to a subject. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 66.7% and the percentage of the mannitol is 33.3%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 25% and the percentage of the mannitol is 75%. Any of the previously described formulations may be administered or used in combination with chemotherapy agents and/or antiemetic drugs.

In some embodiments, to treat and/or prevent nausea and/or vomiting, the present formulation which includes L-Malic acid and mannitol as the active ingredients may be administered or delivered to a subject. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%. Any of the previously described formulations may be administered or used in combination with chemotherapy agents and/or antiemetic drugs.

In some embodiments, to treat and/or prevent nausea and/or vomiting, the present formulation which includes D-Malic acid and mannitol as the active ingredients may be administered or delivered to a subject. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%. Any of the previously described formulations may be administered or used in combination with chemotherapy agents and/or antiemetic drugs.

In some embodiments, to treat and/or prevent nausea and/or vomiting, the present formulation which includes D/L-Malic acid and mannitol as the active ingredients may be administered or delivered to a subject. As a non-limiting example, the percentage of the active ingredient which is mannitol is 25% and the other 75% is D/L-Malic acid. As a non-limiting example, the percentage of the active ingredient which is mannitol is 50% and the other 50% is D/L-Malic acid. As a non-limiting example, the percentage of the active ingredient which is mannitol is 33.3% and the other 66.7% is D/L-Malic acid. As a non-limiting example, the percentage of the active ingredient which is 75% and the other 25% is a D/L-Malic acid. Any of the previously described formulations may be administered or used in combination with chemotherapy agents and/or antiemetic drugs.

In some embodiments, to treat and/or prevent Chemotherapy-induced Nausea and Vomiting (CINV), the present formulation which includes Malic acid (e.g., D-Malic acid, L-Malic acid, D/L-Malic acid) and mannitol as the active ingredients may be administered or delivered to a subject. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 66.7% and the percentage of the mannitol is 33.3%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 25% and the percentage of the mannitol is 75%. Any of the previously described formulations may be administered or used in combination with chemotherapy agents and/or antiemetic drugs.

In some embodiments, to treat and/or prevent Chemotherapy-induced Nausea and Vomiting (CINV), the present formulation which includes L-Malic acid and mannitol as the active ingredients may be administered or delivered to a subject. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%. Any of the previously described formulations may be administered or used in combination with chemotherapy agents and/or antiemetic drugs.

In some embodiments, to treat and/or prevent Chemotherapy-induced Nausea and Vomiting (CINV), the present formulation which includes D-Malic acid and mannitol as the active ingredients may be administered or delivered to a subject. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%. Any of the previously described formulations may be administered or used in combination with chemotherapy agents and/or antiemetic drugs.

In some embodiments, to treat and/or Chemotherapy-induced Nausea and Vomiting (CINV), the present formulation which includes D/L-Malic acid and mannitol as the active ingredients may be administered or delivered to a subject. As a non-limiting example, the percentage of the active ingredient which is mannitol is 25% and the other 75% is D/L-Malic acid. As a non-limiting example, the percentage of the active ingredient which is mannitol is 33.3% and the other 66.7% is D/L-Malic acid. As a non-limiting example, the percentage of the active ingredient which is mannitol is 50% and the other 50% is D/L-Malic acid. As a non-limiting example, the percentage of the active ingredient which is 75% and the other 25% is a D/L-Malic acid. Any of the previously described formulations may be administered or used in combination with chemotherapy agents and/or antiemetic drugs.

In some embodiments, provided are pharmaceutical compositions to treat nausea and/or vomiting comprising (a) one or more perturbation stimuli selected from chemical stimuli, external electrical stimuli, and external mechanical stimuli and (b) one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol.

In some embodiment, provided are orally disintegrating tablets (ODT) to treat nausea and/or vomiting comprising one or more perturbation stimuli selected from chemical stimuli, external electrical stimuli, and external mechanical stimuli and one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol.

In some embodiments, provided are orally disintegrating tablets (ODT) to treat nausea and/or vomiting comprising one or more chemical stimuli and sugar or sugar alcohols. As a non-limiting example, the ODT may include about 40-80% of the at least one chemical stimuli and about 20-60% of the sugar or sugar alcohols. As a non-limiting example, the ODT comprises about 50% of the at least one chemical stimuli and about 50% of the sugar or sugar alcohols. As a non-limiting example, the ODT comprises about 75% of the at least one chemical stimuli and about 25% of the sugar or sugar alcohols.

Provided herein are methods of treating nausea and/or vomiting comprising administering a therapeutically effective amount of the pharmaceutical composition or the ODT described in the preceding paragraphs to a subject in need thereof. The nausea and/or vomiting may be due to a bacterial or virus infection or as a side-effect of medicine or medical therapy. The effects of nausea and/or vomiting is reduced so a subject does not vomit for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) or more than 120 hours ( 5 days).

Provided herein are methods of treating Chemotherapy-induced Nausea and Vomiting (CINV) comprising administering a therapeutically effective amount of the pharmaceutical composition or the ODT described in the preceding paragraphs to a subject in need thereof. The effects of CINV is reduced so a subject does not vomit for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) or more than 120 hours ( 5 days).

### VI. Therapeutic Uses: Salivary Gland Disorders

In some embodiment, the formulations of active ingredients described herein may be used to treat salivary gland disorders.

### Salivary Gland Disorders

In some embodiments, the formulations of active ingredients described herein may be used to treat and/or prevent side-effects of salivary gland disorders. Salivary glands make saliva and release the saliva into the mouth. There are three major salivary glands, (1) parotid glands which are located in the upper part of each cheek close to the ear, (2) submandibular glands which are under the jaw and (3) sublingual glands which are beneath the tongue. Parotid glands release saliva into the inside of the cheek near the molars of the upper jaw. Submandibular glands release saliva behind the lower front teeth. Sublingual glands release saliva onto the floor of the mouth. Besides the three major salivary glands there are between 500-1000 minor salivary glands which are spread throughout the mouth and the throat (e.g., they are located in the back of the throat, back portion of the tongue, inner lips, inner cheeks, palate, pharynx and sinuses). As a non-limiting example, the formulations of active ingredients described herein may be used to treat and/or prevent a reduction in saliva production from any of the salivary glands.

The most common salivary gland disorders include, but are not limited to, Sialolithiasis (salivary gland stones), Sialadenitis (infection of the salivary gland), viral infections causing swelling of the glands (e.g., mumps), cysts (tiny fluid-filled sacs), benign tumors (noncancerous tumors), malignant tumors (cancerous tumors), Sjogren's syndrome (chronic autoimmune disorder), and Sialadenosis (nonspecific salivary gland disorder).

In some embodiments, the formulations of active ingredients described herein may be used to treat and/or prevent salivary gland disorders. In some embodiments, the effect of salivary gland disorders is reduced and a subject is able to produce saliva for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours (4 days), 108 hours, 120 hours ( 5 days) or more than 120 hours ( 5 days).

In some embodiments, to treat and/or prevent salivary gland disorders, the present formulation which includes Malic acid (e.g., D-Malic acid, L-Malic acid, D/L-Malic acid) and/or mannitol as the active ingredient may be administered or delivered to a subject. The active ingredient may be made of 25% to 75% of D-Malic acid, L-Malic acid or D/L-Malic acid. Additionally, the active ingredient may also include 25% to 75% of mannitol. The mannitol may be present in the D or L form or a combination thereof.

In some embodiments, to treat and/or prevent salivary gland disorders, the present formulation which includes Malic acid (e.g., D-Malic acid, L-Malic acid, D/L-Malic acid) and mannitol as the active ingredients may be administered or delivered to a subject. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 67.5% and the percentage of the mannitol is 33.3%. As a non-limiting example, the percentage of the Malic acid (e.g., D-Malic acid, L-Malic acid, D/L-Malic acid) is 25% and the percentage of the mannitol is 75%.

In some embodiments, to treat and/or prevent salivary gland disorders, the present formulation which includes L-Malic acid and mannitol as the active ingredients may be administered or delivered to a subject. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%.

In some embodiments, to treat and/or prevent salivary gland disorders, the present formulation which includes D-Malic acid and mannitol as the active ingredients may be administered or delivered to a subject. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%.

In some embodiments, to treat and/or prevent salivary gland disorders, the present formulation which includes D/L-Malic acid and mannitol as the active ingredients may be administered or delivered to a subject. As a non-limiting example, the percentage of the active ingredient which is mannitol is 25% and the other 75% is D/L-Malic acid. As a non-limiting example, the percentage of the active ingredient which is mannitol is 33.3% and the other 66.7% is D/L-Malic acid. As a non-limiting example, the percentage of the active ingredient which is mannitol is 50% and the other 50% is D/L-Malic acid. As a non-limiting example, the percentage of the active ingredient which is 75% and the other 25% is a D/L-Malic acid.

In some embodiments, provided are pharmaceutical compositions to treat salivary gland disorders comprising (a) one or more perturbation stimuli selected from chemical stimuli, external electrical stimuli, and external mechanical stimuli and (b) one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol. In another aspect, the sugar alcohol is isomaltitol.

In some embodiment, provided are orally disintegrating tablets (ODT) to treat salivary gland disorders comprising one or more perturbation stimuli selected from chemical stimuli, external electrical stimuli, and external mechanical stimuli and one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol. In another aspect, the sugar alcohol is isomaltitol.

In some embodiments, provided are orally disintegrating tablets (ODT) to treat salivary gland disorders comprising one or more chemical stimuli and sugar or sugar alcohols. As a non-limiting example, the ODT may include about 40-80% of the at least one chemical stimuli and about 20-60% of the sugar or sugar alcohols. As a non-limiting example, the ODT comprises about 50% of the at least one chemical stimuli and about 50% of the sugar or sugar alcohols. As a non-limiting example, the ODT comprises about 75% of the at least one chemical stimuli and about 25% of the sugar or sugar alcohols.

Provided herein are methods of treating salivary gland disorders comprising administering a therapeutically effective amount of the pharmaceutical composition or the ODT described in the preceding paragraphs to a subject in need thereof. The effect of salivary gland disorders is reduced and a subject is able to produce saliva for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) or more than 120 hours ( 5 days).

### Side-Effect of Salivary Gland Disorder: Xerostomia

The present disclosure provides compositions and methods of treatment of xerostomia. Xerostomia is dry mouth which results from reduced or absent saliva flow. Xerostomia may be a symptom of a medical condition, a side effect from medication and/or a side effect of medical treatment (e.g., chemotherapy or radiation).

In some embodiments, xerostomia is a side effect of salivary gland disorders. In some embodiments, the salivary gland disorder is Sialolithiasis (salivary gland stones). In some embodiments, the salivary gland disorder is Sialadenitis (infection of the salivary gland). In some embodiments, the salivary gland disorder is viral infections causing swelling of the glands (e.g., mumps). In some embodiments, the salivary gland disorder is cysts (tiny fluid-filled sacs). In some embodiments, the salivary gland disorder is benign tumors (noncancerous tumors). In some embodiments, the salivary gland disorder is malignant tumors (cancerous tumors). In some embodiments, the salivary gland disorder is Sjogren's syndrome (chronic autoimmune disorder). In some embodiments, the salivary gland disorder is Sialadenosis (nonspecific salivary gland disorder).

In some embodiments, the formulations of active ingredients described herein may be used to treat and/or prevent xerostomia. In some embodiments, the effect of xerostomia is reduced and a subject is able to produce saliva for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) or more than 120 hours ( 5 days).

In some embodiments, to treat and/or prevent xerostomia, the present formulation which includes Malic acid (e.g., D-Malic acid, L-Malic acid, D/L-Malic acid) and/or mannitol as the active ingredient may be administered or delivered to a subject. The active ingredient may be made of 25% to 75% of D-Malic acid, L-Malic acid or D/L-Malic acid. Additionally, the active ingredient may also include 25% to 75% of mannitol. The mannitol may be present in the D or L form or a combination thereof. In some embodiments, the formulations may include sugar alcohols such as, but not limited to isomaltitol.

In some embodiments, to treat and/or prevent xerostomia, the present formulation which includes Malic acid (e.g., D-Malic acid, L-Malic acid, D/L-Malic acid) and mannitol as the active ingredients may be administered or delivered to a subject. As a non-limiting example, the percentage of the Malic acid (e.g., D-Malic acid, L-Malic acid, D/L-Malic acid) is 75% and the percentage of the mannitol is 25%. As a non-limiting example, the percentage of the Malic acid (e.g., D-Malic acid, L-Malic acid, D/L-Malic acid) is 66.7% and the percentage of the mannitol is 33.3%. As a non-limiting example, the percentage of the Malic acid (e.g., D-Malic acid, L-Malic acid, D/L-Malic acid) is 50% and the percentage of the mannitol is 50%. As a non-limiting example, the percentage of the Malic acid (e.g., D-Malic acid, L-Malic acid, D/L-Malic acid) is 25% and the percentage of the mannitol is 75%. In some embodiments, the formulations may include sugar alcohols such as, but not limited to isomaltitol.

In some embodiments, to treat and/or prevent xerostomia, the present formulation which includes L-Malic acid and mannitol as the active ingredients may be administered or delivered to a subject. As a non-limiting example, the percentage of the L-Malic acid is 75% and the percentage of the mannitol is 25%. As a non-limiting example, the percentage of the L-Malic acid is 66.7% and the percentage of the mannitol is 33.3%. As a non-limiting example, the percentage of the L-Malic acid is 50% and the percentage of the mannitol is 50%. As a non-limiting example, the percentage of the L-Malic acid is 25% and the percentage of the mannitol is 75%. In some embodiments, the formulations may include sugar alcohols such as, but not limited to isomaltitol.

In some embodiments, to treat and/or prevent xerostomia, the present formulation which includes D-Malic acid and mannitol as the active ingredients may be administered or delivered to a subject. As a non-limiting example, the percentage of the D-Malic acid is 75% and the percentage of the mannitol is 25%. As a non-limiting example, the percentage of the D-Malic acid is 66.7% and the percentage of the mannitol is 33.3%. As a non-limiting example, the percentage of the D-Malic acid is 50% and the percentage of the mannitol is 50%. As a non-limiting example, the percentage of the D-Malic acid is 25% and the percentage of the mannitol is 75%. In some embodiments, the formulations may include sugar alcohols such as, but not limited to isomaltitol.

In some embodiments, to treat and/or prevent xerostomia, the present formulation which includes D/L-Malic acid and mannitol as the active ingredients may be administered or delivered to a subject. As a non-limiting example, the percentage of the active ingredient which is mannitol is 25% and the other 75% is D/L-Malic acid. As a non-limiting example, the percentage of the active ingredient which is mannitol is 33.3% and the other 66.7% is D/L-Malic acid. As a non-limiting example, the percentage of the active ingredient which is mannitol is 50% and the other 50% is D/L-Malic acid. As a non-limiting example, the percentage of the active ingredient which is 75% and the other 25% is a D/L-Malic acid. In some embodiments, the formulations may include sugar alcohols such as, but not limited to isomaltitol.

In some embodiments, provided are pharmaceutical compositions to treat xerostomia comprising (a) one or more perturbation stimuli selected from chemical stimuli, external electrical stimuli, and external mechanical stimuli and (b) one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol. In another aspect, the sugar alcohol is isomaltitol.

In some embodiment, provided are orally disintegrating tablets (ODT) to treat xerostomia comprising one or more perturbation stimuli selected from chemical stimuli, external electrical stimuli, and external mechanical stimuli and one or more excipients. The one or more perturbation stimuli may be a chemical stimuli. The chemical stimulus may include, but are not limited to, Acetic acid, Ascorbic acid, Citric acid, Malic acid (e.g., D-Malic acid, L-Malic acid, D/L-Malic acid), Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof. As a non-limiting example, that the chemical stimuli is Malic acid (e.g., D-Malic acid, L-Malic acid, D/L-Malic acid). As another non-limiting example, the chemical stimuli is Malic acid (e.g., D-Malic acid, L-Malic acid, D/L-Malic acid) and Acetic acid. The pharmaceutical composition which includes Malic acid (e.g., D-Malic acid, L-Malic acid, D/L-Malic acid) and/or acetic acid may also include one or more excipients such as, but not limited to, solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components. As a non-limiting example, the excipient is a sugar or sugar alcohol. The sugar or sugar alcohol may be, but is not limited to, dextrose, dextrin, glucose, isomaltitol, lactitol, lactose, mannitol, sorbitol, sucrose, and xylitol. In one aspect, the sugar alcohol is mannitol. In another aspect, the sugar alcohol is isomaltitol.

In some embodiments, provided are orally disintegrating tablets (ODT) to treat xerostomia comprising one or more chemical stimuli and sugar or sugar alcohols. As a non-limiting example, the ODT may include about 40-80% of the at least one chemical stimuli and about 20-60% of the sugar or sugar alcohols. As a non-limiting example, the ODT comprises about 50% of the at least one chemical stimuli and about 50% of the sugar or sugar alcohols. As a non-limiting example, the ODT comprises about 75% of the at least one chemical stimuli and about 25% of the sugar or sugar alcohols.

Provided herein are methods of treating xerostomia comprising administering a therapeutically effective amount of the pharmaceutical composition or the ODT described in the preceding paragraphs to a subject in need thereof. The effect of xerostomia is reduced and a subject is able to produce saliva for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours (2 days), 60 hours, 72 hours ( 3 days), 84 hours, 96 hours ( 4 days), 108 hours, 120 hours ( 5 days) or more than 120 hours ( 5 days).

### Definitions

At various places in the present specification, substituents of compounds of the present disclosure are disclosed in groups or in ranges. It is specifically intended that the present disclosure include each and every individual subcombination of the members of such groups and ranges.

About: As used herein, the term “about” means +/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biocompatible: As used herein, the term “biocompatible” means compatible with living cells, tissues, organs or systems posing little to no risk of injury, toxicity or rejection by the immune system.

Controlled Release: As used herein, the term “controlled release” refers to a pharmaceutical composition or compound release profile that conforms to a particular pattern of release to effect a therapeutic outcome.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a compound, substance, entity, moiety, cargo or payload to targeted cells.

Dosing regimen: As used herein, a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Feature: As used herein, a “feature” refers to a characteristic, a property, or a distinctive element.

Formulation: As used herein, a “formulation” includes at least one active agent and a delivery agent.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders (such as, but not limited to, natural or synthetic polymers), chelating or complexing agents, coatings, compression aids, disintegrants, dyes (colors) or colorants, emollients, emulsifiers, fillers (diluents), film formers or coatings, flavors, fragrances, glidants (flow enhancers), anticaking agents, lubricants, preservatives, printing inks, releasing agent or modifier, sorbents, surfactants, suspensing or dispersing agents, solubilizing or wetting agents, sweeteners (such as, but not limited to, saccharin, sucralose, aspartame), and waters of hydration. Exemplary excipients include, but are not limited to: butylated hydroxytoluene (BHT), calcium carbonate, calcium phosphate (dibasic), calcium stearate, chitin, chitosan, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sodium stearate, sodium stearyl fumarate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Purified: As used herein, “purify,” “purified,” “purification” means to make substantially pure or clear from unwanted components, material defilement, admixture or imperfection. “Purified” refers to the state of being pure. “Purification” refers to the process of making pure.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention is further illustrated by the following non-limiting examples.

### EXAMPLES

### Example 1

### Preparation of Orally Disintegrating Tablets

Orally disintegrating tablets (ODT) may be made by various methods described in the art. Non-limiting examples of those methods are briefly described below. The methods may be used to make a formulation with any of the active ingredients described herein or taught by Nagar et al. (Journal of Applied Pharmaceutical Science 1(4); 2011: 35-45) and Wah et al. (International Journal of Drug Delivery 2; 2010: 98-107). Non-limiting examples of the active ingredients are Malic acid (e.g., D-Malic acid, L-Malic acid, D/L-Malic acid) and mannitol at a percent of about 75% Malic acid (e.g., D-Malic acid, L-Malic acid, D/L-Malic acid) and about 25% mannitol or about 50% Malic acid (e.g., D-Malic acid, L-Malic acid, D/L-Malic acid) and about 50% mannitol.

### Direct Compression

In one aspect, the ODT may be prepared by a direct compression method. In this method, conventional equipment, commonly available excipients, and a limited number of processing steps are used. It is the most cost-effective table manufacturing technique.

### Freeze Drying or Lyophilization

In one aspect, the ODT may be prepared by a freeze drying method. In this method, the active ingredient is dissolved or dispersed in an aqueous solution of a carrier. The mixture is then poured into a tray with a set of wells of a predetermined size and shape. The tray is then passed through liquid nitrogen freezing the solution with the active ingredient. The frozen wells are placed in refrigerated cabinets to continue the freeze drying before being packaged and shipped. This method provides preparations that are highly porous, have high specific surface area, dissolve rapidly and ultimately show improved absorption and bioavailability.

### Spray Drying

In one aspect, the ODT may be prepared by a spray drying method. In this method, hydrolyzed and non-hydrolyzed gelatins as used as supporting agents, mannitol as a bulking agent, sodium starch glycolate or croscarmellose sodium as disintegrating agent and an acidic agent (e.g., citric acid) and or alkali agent (e.g., sodium bicarbonate) are combined to form a tablet. The prepared tablet is able to disintegrate quickly (e.g., within 20 seconds) when immersed in an aqueous medium.

### Molding

In one aspect, the ODT may be prepared by molding. In this process, water-soluble ingredients with a hydro alcoholic solvent are molded into tablets under pressure lower than that is used in convention tablet compression. This process creates a tablet that is more porous and has enhanced disintegration/dissolution and increased absorption. Dissolution of these tablets can be very rapid (e.g., 5-15 seconds).

### Phase Transition Process

In one aspect, the ODT may be prepared by a phase transition process. In this process, a powder containing two sugar or sugar alcohols with high and low melting points are compressed and heated to a temperature between the melting points. The tablet hardness is increased after the heating process due to the increase of inter particle bond induced by the phase transition of lower melting point sugar or sugar alcohol.

### Melt Granulation

In one aspect, the ODT may be prepared by a melt granulation method. In this method, a hydrophilic waxy binder (super polystate) PEG-6-sterate is integrated with the active agent. The super polystate not only acts as a binder and increases the physical resistance of the tablet but also help the disintegration of the tablet.

### Sublimation

In one aspect, the ODT may be prepared by sublimation. In this process, inert solid ingredients that volatilize rapidly (e.g., urea, camphor ammonium carbonate, ammonium bicarbonate, hexamethylenetetramine) are added to other tablet ingredients and the mixture is compressed into tablets. The volatile materials are removed by sublimation which creates a porous structure.

### Mass Extrusion

In one aspect, the ODT may be prepared by a mass extrusion method. In this method, the active agent is softened using a solvent mixture of water soluble polyethylene glycol, and methanol. Then the softened mass is put through an extruder or syringe to form a cylindrical shape which is then cute into even segments using a heated blade to form tablets.

### Cotton Candy Process

In one aspect, the ODT may be prepared by a cotton candy process. In this process, a matrix of polysaccharides are formed by simultaneous action of flash melting and spinning. The candy floss matrix is then milled and blended with active agents and excipients. This process can accommodate high doses of drug and offered improved mechanical strength.

### Disintegrant Addition

In one aspect, the ODT may be prepared by disintegrant addition. In this method, superdistegrants are added in a desired concentration to the formulation in order to achieve rapid disintegration/dissolution. This method provides tablets which are similar to convention tablets but have a higher percentage of disintegrants, lower hardness and a higher percentage of friability.

### Nanonization

In one aspect, the ODT may be prepared by nanonization. In this method, the size of the drug is reduced to nanosize by milling the drug using a wet-milling technique. The nanocrystals of the drug are stabilized against agglomeration by surface adsorption on selected stabilizers which are then incorporated into ODTs.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A pharmaceutical composition comprising
a. one or more perturbation stimuli selected from chemical stimuli, external electrical stimuli, and external mechanical stimuli; and
b. one or more excipients.

2. The pharmaceutical composition of claim 1, wherein the one or more perturbation stimuli is a chemical stimuli.

3. The pharmaceutical composition of claim 2, wherein the chemical stimuli is selected from the group consisting of Acetic acid, Ascorbic acid, Citric acid, D-Malic acid, L-Malic acid, D/L-Malic acid, Maleic acid, Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof.

4. The pharmaceutical composition of claim 2, wherein the chemical stimuli is D-Malic acid, L-Malic acid, or D/L-Malic acid.

5. (canceled)

6. (canceled)

7. The pharmaceutical composition of claim 2, wherein the chemical stimuli further comprises Acetic acid.

8. The pharmaceutical composition of claim 1, wherein the one or more excipients are selected from the group consisting of solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components.

9. (canceled)

10. The pharmaceutical composition of claim 8, wherein the excipient is a sugar or sugar alcohol, and wherein the sugar or sugar alcohol is selected from the group consisting of dextrose, dextrin, glucose, isomaltitol, lactitol, lactose, mannitol, sorbitol, sucrose, and xylitol.

11. The pharmaceutical composition of claim 10, wherein the sugar or sugar alcohol is a sugar alcohol and where the sugar alcohol is mannitol.

12. An orally disintegrating tablet (ODT) comprising
a. one or more perturbation stimuli selected from chemical stimuli, external electrical stimuli, and external mechanical stimuli; and
b. one or more excipients.

13. The ODT of claim 12, wherein the one or more perturbation stimuli is a chemical stimuli.

14. The ODT of claim 13, wherein the chemical stimuli is selected from the group consisting of Acetic acid, Ascorbic acid, Malic acid, Citric acid, D-Malic acid, L-Malic acid, D/L-Malic acid, Maleic acid, Fumaric acid, Niacin (Vitamin B3), Phosphoric acid, Sodium malate, Sodium citrate, Sodium acetate, Potassium malate, Sodium ascorbate, Calcium citrate, Potassium citrate, Erythorbic acid, Aspartic acid, Prussic acid, Succinic acid, Oxalic acid, Tannic acid, Benzoic acid, Calcium phosphate, and Hydrochloric acid, including all forms, enantiomers, isomers, and analogues thereof.

15. The ODT of claim 13, wherein the chemical stimuli is D-Malic acid, L-Malic acid, or D/L-Malic acid.

16. (canceled)

17. (canceled)

18. The ODT of claim 15, wherein the chemical stimuli further comprises Acetic acid.

19. The ODT of claim 12, wherein the one or more excipients are selected from the group consisting of solvents, diluents, liquid vehicles, dispersion or suspension media or aids, lubricants, surfactants, thickening agents, emulsifying agents, lipids, liposomes, isotonic agents, buffers, gelation agents, preservatives, and other additives, such as colors, sweeteners and flavor components.

20. (canceled)

21. The ODT of claim 19, wherein the excipient is a sugar or sugar alcohol, and wherein the sugar or sugar alcohol is selected from the group consisting of dextrose, dextrin, glucose, isomaltitol, lactitol, lactose, mannitol, sorbitol, sucrose, and xylitol.

22. The ODT of claim 21, wherein the sugar or sugar alcohol is a sugar alcohol and wherein the sugar alcohol is mannitol.

23. The ODT of claim 21, wherein the ODT comprises either (i) about 40-80% of the at least one chemical stimuli and about 20-60% of the sugar or sugar alcohol, (ii) about 50% of the at least one chemical stimuli and about 50% of the sugar or sugar alcohol, (iii) about 75% of the at least one chemical stimuli and about 25% of the sugar or sugar alcohol, or (iv) about 66.7% of the at least one chemical stimuli and about 33.3% of the sugar or sugar alcohol.

24. (canceled)

25. (canceled)

26. (canceled)

27. A method of treating a disease, disorder or condition related to respiratory arrythmia comprising administering a therapeutically effective amount of the pharmaceutical composition of claim 1 to a subject in need thereof.

28. The method of claim 27, wherein the respiratory arrythmia is caused by a disease, disorder or condition selected from the group consisting of cancer, advanced cancer, apnea, orthopnea, dyspnea, hyperpnea, hyperventilation, hypoventilation, tachypnea, Kussmaul respiration, Cheyne-Stokes respiration, sighing respiration, Biot respiration, apneustic breathing, central neurogenic hyperventilation, central neurogenic hypoventilation, agonal breathing, allergies, multiple sclerosis, Parkinson disease, Trauma, Postpolio syndrome, Amyotrophic Lateral Sclerosis, Guillain-Barre syndrome, Charcot-Marie-Tooth disease, Myasthenia gravis, Botulism, Duchenne muscular dystrophy, Polymyositis/dermatomyositis, Postparalysis myopathy, asthma, chronic obstructive pulmonary disease (COPD) and interstitial lung disease, anxiety related disorders, gastroesophageal reflux disease (GERD), panic disorder, Rett syndrome, effects of post-surgery recovery, weight loss, and insomnia.

29. The method of claim 28, wherein the disease, disorder or condition related to respiratory arrythmia is chronic hiccups.

30. The method of claim 29, wherein the effect of chronic hiccups is reduced so a subject does not hiccup for a time period selected from the group consisting of 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours ( 1 day), 36 hours, 48 hours ( 2 days), 60 hours, 72 hours ( 3 days), 84 hours, 96 hours ( 4 days), 108 hours, 120 hours ( 5 days) and more than 120 hours ( 5 days).
